(19) United States
(12) Patent Application Publication
(10) Pub. No.: US 2024/0398817 A1
(43) Pub. Date: Dec. 5, 2024

(54) CARDIO- AND RENOSAFE ANTIDIABETIC THERAPY

(71) Applicant: Boehringer Ingelheim International GmbH, Ingelheim am Rhein (DE)

(72) Inventors: Maximilian VON EYNATTEN, Erding (DE); Odd-Erik JOHANSEN, Hovik (NO)

(21) Appl. No.: 18/800,173

(22) Filed: Aug. 12, 2024

Related U.S. Application Data
(63) Continuation of application No. 17/666,639, filed on Feb. 8, 2022.
Application No. 17/666,639 is a continuation of application No. 16/512,432, filed on Jul. 16, 2019, now abandoned.

(30) Foreign Application Priority Data
Jul. 17, 2018 (EP) 18 184 034.9
Aug. 3, 2018 (EP) 18 187 272.2
Sep. 28, 2018 (EP) 18 197 472.6
Oct. 26, 2018 (EP) 18 202 843.1
May 29, 2019 (EP) 19 177 388.6

(51) Int. Cl. A61K 31/522; A61K 9/00; A61P 3/10

(52) U.S. Cl. CPC A61K 31/522; A61K 9/0053; A61P 3/10

(57) ABSTRACT

The present invention relates to cardio- and renosafe antidiabetic therapy.

### FIELD OF THE INVENTION

The present invention relates to a certain DPP-4 inhibitor, preferably linagliptin (optionally in combination with one or more other active agents) for use in cardiovascular-and/or renal-safe antidiabetic treatment of diabetes (preferably type 2 diabetes) patients and/or to provide certain micro-and/or macrovascular benefits in these patients, including in (human) patients with or at-risk of (micro-and/or macro-) vascular diseases, such as e.g. patients having or being at-risk of cardiovascular and/or microvascular (e.g. renal/kidney) diseases, such as e.g. patients at high or increased vascular (cardio-renal) risk, such as e.g. patients at high or increased risk of cardiovascular and/or renal events or complications.

In an embodiment, patients (especially type 2 diabetes patients) at high vascular risk include patients with high cardiovascular risk, the majority of whom also have kidney disease (CKD, an important risk factor for cardiovascular disease).

Accordingly, in an embodiment, patients (especially type 2 diabetes patients) at high vascular (cardio-renal) risk include patients having kidney disease (CKD) and/or albuminuria and/or impaired renal function (an “unmet-medical need population” of patients where the conventional antidiabetic treatment armamentarium is label restricted, particularly at advanced stage). For example, patients of this embodiment are with prevalent CKD and moderate to severe kidney dysfunction such as having eGFR <45 ml/min/1.73m² or eGFR <30 ml/min/1.73m². For further example, patients according to the present invention are with prevalent CKD and/or micro-or macro-albuminuria such as having UACR 30-300 mg/g or UACR >300 mg/g, respectively. For yet further example, patients according to the present invention have both impaired renal function (such as mild, moderate, moderate/severe or severe renal impairment) and micro-or macro-albuminuria.

### BACKGROUND OF THE INVENTION

People with type 2 diabetes (T2D) are at increased risk for both cardiovascular (CV) disease and microvascular complications such as chronic kidney disease (CKD) and kidney failure/renal impairment. In 2008, concerns about adverse CV events associated with the peroxisome proliferator-activated receptor agonists rosiglitazone and muraglitazar were among the issues that led the US Food and Drug Administration (FDA) and European Medicines Agency (EMA) to mandate that novel glucose-lowering drugs for treatment of T2D demonstrate CV safety. The CV outcome trials conducted in response to this guidance over the past decade have consequently focused on T2D patients at high risk for CV complications. In contrast, evaluation of novel glucose-lowering drugs in individuals at high risk of adverse kidney outcomes has been sparse and relatively neglected.

Approximately 50% of patients with T2D globally also have some evidence of CKD, which is associated with significantly increased risk of progression to endstage kidney disease (ESKD) and premature mortality. CKD is also one of the strongest risk factors for CV events. A 2016 summit convened by the International Society of Nephrology concluded that a concerted effort is required to increase the quantity and quality of clinical trials investigating CKD; however, there are notable challenges involved in conducting such studies. The paucity of clinical trials specifically designed to evaluate kidney-related efficacy and safety outcomes with glucose-lowering drugs represents an important gap in knowledge to support informed treatment decision-making in patients with T2D at high risk for kidney complications.

Dipeptidyl peptidase-4 (DPP-4) inhibitors are now established as oral glucose-lowering drugs with little intrinsic risk of causing hypoglycemia or weight gain. The DPP-4 inhibitors evaluated to date in CV outcomes studies (saxagliptin, alogliptin, sitagliptin) have demonstrated CV safety with regard to atherosclerotic CV disease outcomes, with neutral effects on major adverse CV events compared with placebo. However, the incidence of hospitalization for heart failure was statistically increased in the SAVOR-TIMI 53 trial of saxagliptin versus placebo and numerically increased in the EXAMINE trial of alogliptin versus placebo; whereas no effect on the incidence of heart failure hospitalization was observed in the TECOS trial of sitagliptin versus placebo. These observations have prompted FDA product label warnings in the US for all members of the DPP-4 inhibitor class.

Notably, these previous CV outcomes studies (saxagliptin, alogliptin, sitagliptin) enrolled only limited numbers of people with type 2 diabetes and concomitant chronic kidney disease (CKD), a group of patients with a much higher CV risk and limited treatment options due to renal impairment (particularly at advanced stage). Patients with advanced CKD have been largely excluded from previous CV outcomes studies of glucose-lowering drugs, resulting in scarity of available safety information for this particular population.

Therefore there is need for further antidiabetic treatments which are efficacious, well tolerated, easy to be used (e.g. independent from patients' kidney function), and which have both a safe CV and a safe kidney clinical profile, especially including in at-risk patients such as having or being at increased or high risk of both CV and kidney complications (such as e.g. patients who have evidence of compromised kidney function (CKD, renal impairment) with or without CV disease).

### SUMMARY OF THE INVENTION

Within the scope of the present invention it has now been found that the certain DPP-4 inhibitor, preferably linagliptin, optionally in combination with one or more other active agents as defined herein, has properties, which make it useful for the purpose of this invention and/or for fulfilling one or more of the needs mentioned herein.

Linagliptin (5 mg once daily) shows long-term clinical safety (both cardiovascular and renal) as well as certain benefits (e.g. reduction of albuminuria, improvements in microvascular renal and eye outcomes) in a Cardiovascular and Renal Outcomes Trial (assessing cardiovascular safety and kidney/renal microvascular outcome in patients with type 2 diabetes at high or increased vascular risk), even in those patients most vulnerable for vascular complications (i.e. patients at high cardio-renal risk, such as patients having or at high risk for CV/heart and/or kidney/renal disease, such as defined herein, e.g. cf. Condition I, Condition II, such as e.g. wherein the (cardio-renal) risk is based on (history of) established macrovascular disease and/or renal disease).

This Cardiovascular and Renal Outcomes Trial has been designed to assess CV and kidney/renal microvascular outcomes of linagliptin (5 mg once daily) versus placebo (each when added to standard care) in adults with type 2 diabetes and established CV and/or kidney complications.

Standard of care includes both glucose lowering agents and cardiovascular drugs (including antihypertensive and lipid lowering agents).

Compared with the spectrum of CV outcome trials conducted in patients with type 2 diabetes to date, the present Cardiovascular and Renal Outcomes Trial has the highest number of individuals with prevalent kidney disease, including a large proportion of patients with severe kidney impairment (e.g. impaired kidney function with glomerular filtration rate below 30 mL/min/m²) and/or elevated albuminuria. These individuals are at high cardio-renal risk, face limited glucose-lowering treatment options and have been largely underrepresented in previous CV outcome trials in type 2 diabetes. This population also reflects patients that doctors see in their daily practice.

Importantly, it has been found from the present Cardiovascular and Renal Outcomes Trial that in adults with type 2 diabetes and high cardiovascular risk, the majority of whom also have kidney disease (a population that has previously been underrepresented in other cardiovascular outcomes trials in diabetes), linagliptin demonstrates similar cardiovascular safety compared to placebo.

Whereas in the US label of two members of the DPP-4 inhibitors class an increased risk of hospitalisation for heart failure is included, linagliptin shows no increased risk of hospitalization for heart failure.

In addition, it has been found from the present Cardiovascular and Renal Outcomes Trial that in adults with type 2 diabetes and high cardiovascular risk, the majority of whom also have kidney disease (a population that has previously been underrepresented in other cardiovascular outcomes trials in diabetes), linagliptin demonstrates similar renal/kidney safety compared to placebo.

Furthermore, it has been found from the present Cardiovascular and Renal Outcomes Trial that linagliptin reduces albuminuria as well as HbA1c, without increasing the risk for hypoglycaemia. Further, it has been found from the present Cardiovascular and Renal Outcomes Trial that linagliptin improves microvascular renal and eye outcomes.

The patients of this Cardiovascular and Renal Outcomes Trial assessing cardiovascular safety and renal microvascular outcome with linagliptin in patients with type 2 diabetes at high vascular risk have been treated with 5 mg linagliptin once daily (on top of standard of care) and observed for a median duration of 2.2 years.

### BRIEF DESCRIPTION OF THE DRAWINGS

FIG. 1 (Time to First Occurrence of (3P) MACE in this Cardiovascular and Renal Outcomes trial) shows time to first occurrence of three point (3P) MACE (3P-MACE, major adverse cardiac event defined as a cardiovascular death or a nonfatal myocardial infarction (MI) or a nonfatal stroke in this Cardiovascular and Renal Outcomes trial.

FIG. 2 shows effects of linagliptin (LINA) vs placebo (PBO) on individual and composite heart failure (HF)-related outcomes, recurrent hospitalization for heart failure (hHF) events, initiation of diuretic therapy and in subgroups of interest, in this Cardiovascular and Renal Outcomes trial.

FIG. 3A shows changes over time in glycated hemoglobin levels (mean±SE) in this Cardiovascular and Renal Outcomes trial. Changes from baseline in glycated hemoglobin levels were calculated with the use of a repeated-measures analysis as a mixed model. The model included baseline glycated 10 hemoglobin as a linear covariate, with baseline estimated glomerular filtration rate, geographic region, randomized treatment, visit, visit by randomized treatment interaction, and baseline glycated hemoglobin by visit interaction as fixed effects.

FIG. 3B shows incidence rate of hypoglycemia in this Cardiovascular and Renal Outcomes trial.

Shown are incidence rates of any investigator reported hypoglycemic event, investigator reported hypoglycemic event with plasma glucose <54 mg/dl or severe event, or severe hypoglycemic events. Severe events defined as events requiring assistance of another person to actively administer carbohydrate, glucagon or other resuscitative actions.

FIG. 3C shows glucose lowering drugs introduced post-baseline in this Cardiovascular and Renal Outcomes trial. Shown are percentage of patients with glucose-lowering medication initiated after first trial administration and without previous (either ongoing or discontinued) prescription of the same preferred name. Dose increases are not considered. Hazard ratios (HR) for time to first initiation of the corresponding antidiabetic medication are based on a Cox regression model.

FIG. 3D shows initiation or dose increase of insulin in this Cardiovascular and Renal Outcomes trial.

Kaplan-Meier estimates and HR (95% confidence interval) for time to initiation or dose increase of insulin. Initiation of insulin was considered if continuous period of insulin ≥3 months. Insulin dose increase was defined as an increase for at least 3 months of>50%;>30%; >20% for patients with baseline daily insulin dose of ≤10 units; >10 and ≤20 units; >20 units, respectively.

FIGS. 4A to 4D show primary and further cardiovascular outcomes, in this Cardiovascular and Renal Outcomes trial:

FIG. 4A shows time to first occurrence of 3P-MACE,

FIG. 4B shows time to first occurrence of cardiovascular (CV) death,

FIG. 4C shows time to first occurrence of all-cause death,

FIG. 4D shows time to first occurrence of hospitalization for heart failure.

FIGS. 5A to 5D show key secondary outcome and further microvascular outcomes in this Cardiovascular and Renal Outcomes trial:

FIG. 5A shows time to first occurrence of kidney composite outcome,

FIG. 5B shows time to first occurrence of renal death or sustained end stage kidney disease,

FIG. 5C shows time to first occurrence of albuminuria progression,

FIG. 5D shows time to first occurrence of composite microvascular endpoint.

FIG. 6 shows hypoglycemia rates in subgroups of patients at elevated hypoglycemia risk in this Cardiovascular and Renal Outcomes trial.

### DETAILED DESCRIPTION OF THE INVENTION

In more detail, the following findings have been made:

### Cardiovascular and Renal Outcomes Trial

### Efficacy:

The effect of linagliptin on cardiovascular risk in adult patients with type 2 diabetes mellitus and with increased CV risk evidenced by a history of established macrovascular or renal disease (e.g. as defined herein) was evaluated in a multi-center, multi-national, randomized, double-blind parallel group trial. The trial compared the risk of experiencing a major adverse cardiovascular event (MACE) between linagliptin and placebo when these were added to and used concomitantly with standard of care treatments for diabetes (HbA1c), cardiovascular risk factors and renal disease. The trial was event driven and patients were followed until at least 611 primary outcome events accrued.

A total of 6979 patients were treated (linagliptin 5 mg=3494; placebo=3485) and followed for a median of 2.2 years (median time on treatment 1.9 years). Approximately 80% of the study population was Caucasian, 9% was Asian, and 6% was Black. The mean age was 66 years and 63% were male.

The mean HbA1c at baseline was 8.0% and participants had a mean duration of type 2 diabetes mellitus of approximately 15 years, further 10% were current smokers. The trial population included 1211 (17.4%) patients ≥75 years of age and 4348 (62.3%) patients with renal impairment. Approximately 19% of the population had moderate renal impairment (eGFR ≥45 to <60 mL/min/1.73 m²), 28% of the population had moderately severe renal impairment (eGFR ≥30 to <45 mL/min/1.73 m²) and 15% had severe renal impairment (eGFR <30 mL/min/1.73 m²). Overall, the use of diabetes medications was balanced across treatment groups (metformin 54%, sulfonylurea 32%, and insulin 57%). The use of medications to reduce cardiovascular risk was also balanced (aspirin 62%, statins 71%, ACE inhibitors or ARBs 81%, beta blockers 60%, and calcium channel blockers 41%).

The primary endpoint in this trial was the time to first occurrence of three point (3P) MACE. A major adverse cardiac event was defined as a cardiovascular death or a nonfatal myocardial infarction (MI) or a nonfatal stroke. The statistical analysis plan tested for non-inferiority for the occurrence of (3P) MACE. If non-inferiority was demonstrated the hierarchical testing strategy included superiority on (3P) MACE and a renal composite in parallel. The secondary endpoint was a renal composite, defined as renal death or sustained end stage renal disease or sustained decrease of 40% or more in eGFR.

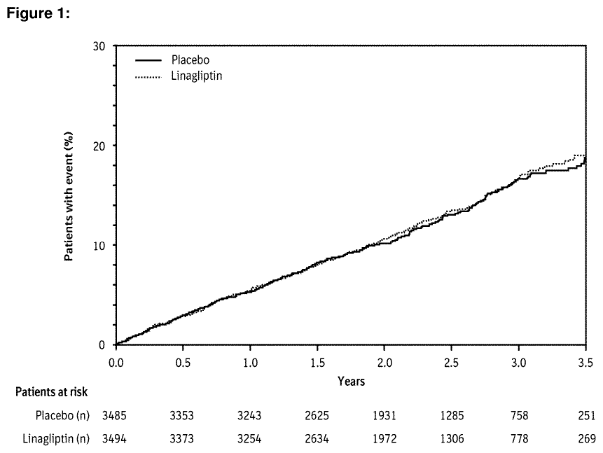

After a median follow up of 2.2 years (median time on treatment 1.9 years), linagliptin, when added to standard of care, did not increase the risk of major adverse cardiovascular events (MACE) or renal outcome events (Table 1+Table 2 and FIG. 1).

The results of the primary endpoint (composite of first event of CV death, non-fatal MI or non-fatal stroke (MACE)) of this trial are shown in Table 1 and FIG. 1. The incidence of (3P) MACE was similar in both treatment arms; placebo (56.3 MACE per 1000 patient years) and linagliptin (57.7 MACE per 1000 patient years). The estimated hazard ratio of MACE associated with linagliptin relative to placebo was 1.02 (95% CI; 0.89, 1.17). The upper bound of this confidence interval 1.17, excluded a pre-defined risk margin larger than 1.3.

TABLE 1
Major Adverse Cardiovascular Events (MACE) by Treatment Group in this
Cardiovascular and Renal Outcomes trial

| | Linagliptin 5 mg | | Placebo | | |
|---|---|---|---|---|---|
| | n = 3494 | | n = 3485 | | |
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| Primary CV | 434 (12.4) | 57.7 | 420 (12.1) | 56.3 | 1.02 (0.89, 1.17) |
| composite (CV | | | | | |
| death, non-fatal | | | | | |
| MI, non-fatal stroke) | | | | | |

*PY = patient years

In this trial, there was no increase in the risk of hospitalization for heart failure, which was an additional adjudicated event. The estimated hazard ratio of hospitalization for heart failure associated with linagliptin relative to placebo was 0.90 (95% CI; 0.74, 1.08). In the trial 209 (6.0%) patients treated with linagliptin and 226 (6.5%) patients treated with placebo were hospitalized for heart failure.

Vital status was obtained for 99.7% of subjects in the trial. A total of 740 deaths were recorded during this trial (Table 3). Of these deaths, 70% were adjudicated as cardiovascular deaths. The risk of deaths from all cause was not statistically different between the treatment groups (HR: 0.98; 95% CI: 0.84, 1.13).

TABLE 3
Mortality by Treatment Group in this
Cardiovascular and Renal Outcomes trial

| | Linagliptin 5 mg | | Placebo | | |
|---|---|---|---|---|---|
| | n = 3494 | | n = 3485 | | |
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| All- | 367 (10.5%) | 46.9 | 373 (10.7%) | 48.0 | 0.98 |
| cause | | | | | (0.84, 1.13) |
| mortal- | | | | | |
| ity | | | | | |
| CV | 255 (7.3%) | 32.6 | 264 (7.6%) | 34.0 | 0.96 |
| death | | | | | (0.81, 1.14) |

The incidence of the renal composite (defined as renal death or sustained end stage renal disease or sustained decrease of 40% or more in eGFR) was similar in both treatment arms; placebo (46.6 renal composite per 1000 patient years) and linagliptin (48.9 renal composite per 1000 patient years). The estimated hazard ratio of the renal composite associated with linagliptin relative to placebo was 1.04 (95% CI; 0.89, 1.22).

TABLE 2
Renal outcome events by Treatment Group in this Cardiovascular and Renal Outcomes trial

| | Linagliptin 5 mg | | Placebo | | |
|---|---|---|---|---|---|
| | n = 3494 | | n = 3485 | | |
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| Secondary renal | 327 (9.4) | 48.9 | 306 (8.8) | 46.6 | 1.04 (0.89, 1.22) |
| composite (renal | | | | | |
| death, ESRD, | | | | | |
| 40% sustained | | | | | |
| decrease in eGFR) | | | | | |

*PY = patient years

In analyses for albuminuria progression (change from normal albuminuria to micro-or macroalbuminuria, or from microalbuminuria to macroalbuminuria) a hazard ratio of 0.86 (95% CI 0.78, 0.95) was observed for linagliptin versus placebo.

The estimated hazard ratio for time to first occurrence for the composite microvascular endpoint (of renal and eye outcomes) was 0.86 (95% CI 0.78, 0.95) for linagliptin versus placebo; mainly driven by albuminuria progression. The microvascular endpoint of renal and eye outcomes was defined as the composite of renal death, sustained ESRD, sustained decrease of ≥50% in eGFR, albuminuria progression, use of retinal photocoagulation or intravitreal injections of an anti-VEGF therapy for diabetic retinopathy or vitreous hemorrhage or diabetes-related-blindness.

### Safety:

This outcome study evaluated the cardiovascular and renal safety of linagliptin versus placebo in patients with type 2 diabetes and with increased CV risk evidenced by a history of established macrovascular or renal disease. The study included 3494 patients treated with linagliptin (5 mg) and 3485 patients treated with placebo. Both treatments were added to standard of care targeting regional standards for HbA1c and CV risk factors. Safety data from this study was in line with previous known safety profile of linagliptin. The overall incidence of adverse events and serious adverse events in patients receiving linagliptin was similar to that in patients receiving placebo. No new safety findings were observed.

In the treated population, severe hypoglycaemic events (requiring assistance) were reported in 3.0% patients on linagliptin and in 3.1% on placebo. Among patients who were using sulfonylurea at baseline, the incidence of severe hypoglycaemia was 2.0% in linagliptin-treated patients and 1.7% in placebo treated patients. Among patients who were using insulin at baseline, the incidence of severe hypoglycaemia was 4.4% in linagliptin-treated patients and 4.9% in placebo treated patients.

In the overall study observation period adjudicated acute pancreatitis was reported in 9 (0.3%) patients treated with linagliptin and in 5 (0.1%) patients treated with placebo.

In this study, bullous pemphigoid was reported in 7 (0.2%) patients treated with linagliptin and in no patient treated with placebo.

### Conclusions:

This trial evaluated the effect of linagliptin on cardiovascular and kidney outcomes in patients with type 2 diabetes who were at high cardiovascular risk. Unlike other completed CV outcome trials with DPP-4 inhibitors, this trial included a particularly high proportion of patients with prevalent kidney disease in addition to those with established macrovascular disease, thereby investigating a highly vulnerable population for cardiovascular and renal events. In this trial, linagliptin was shown to be non-inferior to placebo on top of standard of care for time to first occurrence of CV death, non-fatal MI, or non-fatal stroke (3P-MACE). There was also no increased risk for hospitalisation for heart failure or any other heart failure endpoint. Linagliptin was comparable to placebo in time to first occurrence of renal death, sustained ESRD or sustained decrease of 40% or more in eGFR from baseline. Linagliptin reduced albuminuria as well as HbA1c, without increasing the risk for hypoglycaemia. Linagliptin was well tolerated overall and the safety profile in this study was consistent with the known profile of the drug. In summary, cardiovascular and renal safety of linagliptin have been demonstrated in a CV high risk population with established macrovascular and/or prevalent kidney disease.

### Accordingly:

The present invention relates to linagliptin, optionally in combination with one or more other active agents, for use in the treatment of diabetic (preferably type 2 diabetes) patients wherein linagliptin effects the treatment without increasing the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke.

The present invention relates to linagliptin, optionally in combination with one or more other active agents, for use in the treatment of diabetic (preferably type 2 diabetes) patients, wherein the treatment with linagliptin results in a risk of the three point major adverse cardiovascular events (3P-MACE) as shown in Table 1 of the description, such as e.g. resulting in a hazard ratio (HR) of 1.02 (95% CI; 0.89, 1.17) for the risk of three point major adverse cardiovascular events (3P-MACE) relative to treatment with placebo.

The present invention relates to linagliptin, optionally in combination with one or more other active agents, for use in the treatment of diabetic (preferably type 2 diabetes) patients wherein linagliptin effects the treatment without increasing the risk of hospitalization for heart failure.

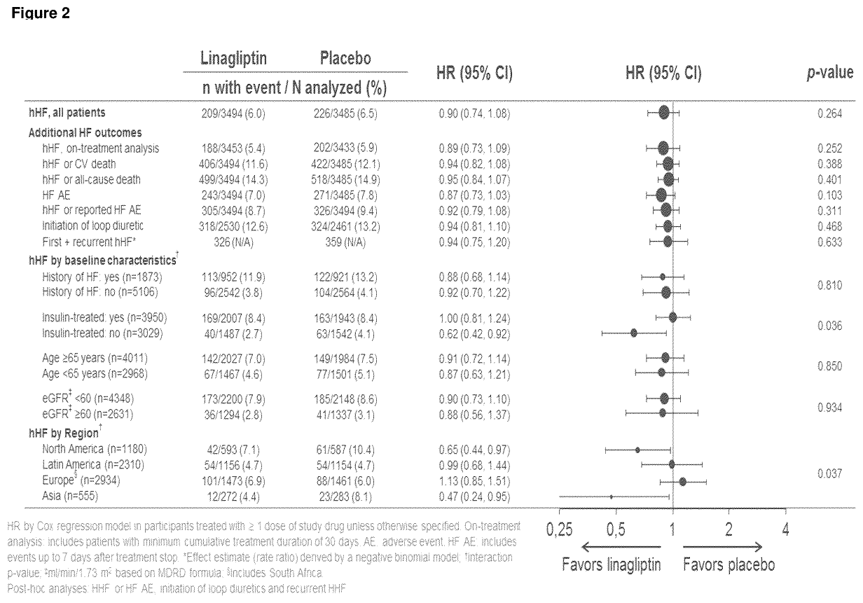

The present invention relates to linagliptin, optionally in combination with one or more other active agents, for use in the treatment of diabetic (preferably type 2 diabetes) patients, wherein the treatment with linagliptin results in a risk for the hospitalization for heart failure as shown in FIG. 2 of the description, such as e.g. resulting in a hazard ratio (HR) of 0.90 (95% CI; 0.74, 1.08) for the risk of hospitalization for heart failure relative to treatment with placebo.

The present invention relates to linagliptin, optionally in combination with one or more other active agents, for use in the treatment of diabetic (preferably type 2 diabetes) patients wherein linagliptin effects the treatment without increasing the risk of key renal outcome events, wherein the key renal outcome events include renal death, sustained end stage renal disease (ESRD) and/or sustained decrease of 40% or more in estimated glomerular filtration rate (eGFR).

The present invention relates to linagliptin, optionally in combination with one or more other active agents, for use in the treatment of diabetic (preferably type 2 diabetes) patients, wherein the treatment with linagliptin results in a risk of the key renal outcome events as shown in Table 2 of the description, such as e.g. resulting in a hazard ratio (HR) of 1.04 (95% CI; 0.89, 1.22) for the risk of renal outcome events relative to treatment with placebo.

The present invention relates to linagliptin, optionally in combination with one or more other active agents, for use in the treatment of diabetes (preferably type 2 diabetes) in patients in need thereof, wherein the treatment is characterized in that:

i) linagliptin does not increase the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke, ii) linagliptin does not increase the risk of hospitalization for heart failure, and/or iii) linagliptin does not increase the risk of key renal outcome events, wherein the key renal outcome events include renal death, sustained end stage renal disease (ESRD) and/or sustained decrease of 40% or more in estimated glomerular filtration rate (eGFR).

The present invention relates to linagliptin, optionally in combination with one or more other active agents, for use in the treatment of diabetic (preferably type 2 diabetes) patients wherein linagliptin effects the treatment without increasing the risk of deaths from all cause (all-cause mortality).

The present invention relates to linagliptin, optionally in combination with one or more other active agents, for use in the treatment of diabetic (preferably type 2 diabetes) patients, wherein the treatment with linagliptin results in a risk of all-cause mortality as shown in Table 3 of the description, such as e.g. resulting in a hazard ratio (HR) of 0.98 (95% CI; 0.84, 1.13) for all-cause mortality relative to treatment with placebo.

The present invention relates to linagliptin, optionally in combination with one or more other active agents, for use in the treatment of diabetic (preferably type 2 diabetes) patients wherein linagliptin effects the treatment without increasing the risk of deaths from cardiovascular cause (CV death).

The present invention relates to linagliptin, optionally in combination with one or more other active agents, for use in the treatment of diabetic (preferably type 2 diabetes) patients, wherein the treatment with linagliptin results in a risk of CV death as shown in Table 3 of the description, such as e.g. resulting in a hazard ratio (HR) of 0.96 (95% CI; 0.81, 1.14) for CV death relative to treatment with placebo.

The present invention relates to linagliptin, optionally in combination with one or more other active agents, for use in the treatment of diabetic (preferably type 2 diabetes) patients wherein said linagliptin treatment does not result (e.g. at 2.2. years) in a hazard ratio (HR) for risk of 3 point major adverse cardiovascular events (3P-MACE) that is significantly greater than 1 (e.g. 95% confidence interval for the HR for risk of 3P-MACE of 0.89 to 1.17) relative to placebo treatment, wherein the 3 point major adverse cardiovascular events (3P-MACE) include cardiovascular death, nonfatal myocardial infarction (MI) and/or nonfatal stroke.

The present invention relates to linagliptin, optionally in combination with one or more other active agents, for use in the treatment of diabetic (preferably type 2 diabetes) patients wherein said linagliptin treatment does not result (e.g. at 2.2. years) in a hazard ratio (HR) for risk of key renal outcome events that is significantly greater than 1 (e.g. 95% confidence interval for the HR for risk of key renal outcome events of 0.89 to 1.22) relative to placebo treatment, wherein the key renal outcome events include renal death, sustained end stage renal disease (ESRD) and/or sustained decrease of 40% or more in estimated glomerular filtration rate (eGFR).

The present invention relates to linagliptin, optionally in combination with one or more other active agents, for use in the treatment of diabetic (preferably type 2 diabetes) patients wherein said linagliptin treatment results (e.g. at 2.2. years) in a numerical reduction in the rate of hospitalization for heart failure and/or does not result in a hazard ratio (HR) for risk of hospitalization for heart failure that is significantly greater than 1 (e.g. 95% confidence interval for the HR for risk of hospitalization for heart failure of 0.74 to 1.08) relative to placebo treatment.

The present invention relates to linagliptin, optionally in combination with one or more other active agents, for use in the treatment of diabetic (preferably type 2 diabetes) patients wherein said linagliptin treatment results (e.g. at 2.2. years) in a numerical reduction in the rate of deaths from all cause and/or does not result in a hazard ratio (HR) for risk of deaths from all cause that is significantly greater than 1 (e.g. 95% confidence interval for the HR for risk of all-cause mortality of 0.84 to 1.12) relative to placebo treatment.

The present invention relates to linagliptin, optionally in combination with one or more other active agents, for use in the treatment of diabetic (preferably type 2 diabetes) patients wherein said linagliptin treatment results (e.g. at 2.2. years) in a numerical reduction in the rate of cardiovascular deaths and/or does not result in a hazard ratio (HR) for risk of cardiovascular deaths that is significantly greater than 1 (e.g. 95% confidence interval for the HR for risk of CV death of 0.81 to 1.14) relative to placebo treatment.

The present invention relates to linagliptin, optionally in combination with one or more other active agents, for use in the treatment of diabetic (preferably type 2 diabetes) patients wherein linagliptin effects the treatment with reducing the risk of albuminuria progression, wherein the albuminuria progression includes change from normal albuminuria to micro-or macroalbuminuria and/or change from microalbuminuria to macroalbuminuria.

The present invention relates to linagliptin, optionally in combination with one or more other active agents, for use in the treatment of diabetic (preferably type 2 diabetes) patients wherein said linagliptin treatment results 8 (e.g. at 2.2. years) in a numerical reduction in the rate of albuminuria progression and/or in a hazard ratio (HR) for risk of albuminuria progression that is significantly lower than 1 (e.g. 95% confidence interval for the HR for risk of albuminuria progression of 0.78 to 0.95, such as e.g. 0.86) relative to placebo treatment, wherein the albuminuria progression includes change from normal albuminuria to micro-or macroalbuminuria and/or change from microalbuminuria to macroalbuminuria.

The present invention relates to linagliptin, optionally in combination with one or more other active agents, for use in the treatment of diabetic (preferably type 2 diabetes) patients wherein linagliptin effects the treatment with reducing the risk of albuminuria progression, wherein the albuminuria progression includes change from normal albuminuria to micro-or macroalbuminuria and/or change from microalbuminuria to macroalbuminuria, wherein said risk of albuminuria progression is reduced from about 5% to about 25% or about 10% to about 20% compared to placebo, such as reduced about 14% compared to placebo.

The present invention relates to linagliptin, optionally in combination with one or more other active agents, for use in the treatment of diabetic (preferably type 2 diabetes) patients wherein linagliptin effects the treatment with reducing the risk of microvascular renal and/or eye complications, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness.

The present invention relates to linagliptin, optionally in combination with one or more other active agents, for use in the treatment of diabetic (preferably type 2 diabetes) patients wherein said linagliptin treatment results (e.g. at 2.2. years) in a numerical reduction in the rate of microvascular renal and/or eye complications and/or in a hazard ratio (HR) for risk of microvascular renal and/or eye complications that is significantly lower than 1 (e.g. 95% confidence interval for the HR for risk of albuminuria progression of 0.78 to 0.95, such as e.g. 0.86) relative to placebo treatment, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness.

The present invention relates to linagliptin, optionally in combination with one or more other active agents, for use in the treatment of diabetic (preferably type 2 diabetes) patients wherein linagliptin effects the treatment with reducing the risk of microvascular renal and/or eye complications, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness, wherein said risk of microvascular renal and/or eye complications is reduced from about 5% to about 25% or about 10% to about 20% compared to placebo, such as reduced about 14% compared to placebo.

The present invention relates to linagliptin, optionally in combination with one or more other active agents, for use in the treatment of diabetes (preferably type 2 diabetes) in patients in need thereof, wherein the treatment is characterized in that:
i) linagliptin does not increase the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke,
ii) linagliptin does not increase the risk of hospitalization for heart failure,
iii) linagliptin does not increase the risk of key renal outcome events, wherein the key renal outcome events include renal death, sustained end stage renal disease (ESRD) and/or sustained decrease of 40% or more in estimated glomerular filtration rate (eGFR), and/or
iv) linagliptin prevents or reduces the risk of albuminuria progression, wherein the albuminuria progression includes change from normal albuminuria to micro-or macroalbuminuria and/or change from microalbuminuria to macroalbuminuria.

The present invention relates to linagliptin, optionally in combination with one or more other active agents, for use in the treatment of diabetes (preferably type 2 diabetes) in patients in need thereof, wherein the treatment is characterized in that:
i) linagliptin does not increase the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke,
ii) linagliptin does not increase the risk of hospitalization for heart failure,
iii) linagliptin does not increase the risk of key renal outcome events, wherein the key renal outcome events include renal death, sustained end stage renal disease (ESRD) and/or sustained decrease of 40% or more in estimated glomerular filtration rate (eGFR),
iv) linagliptin prevents or reduces the risk of albuminuria progression, wherein the albuminuria progression includes change from normal albuminuria to micro-or macroalbuminuria and/or change from microalbuminuria to macroalbuminuria, and/or
v) linagliptin prevents or reduces the risk of microvascular renal and/or eye complications, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness.

Linagliptin, optionally in combination with one or more other active agents, for use in the treatment of a diabetic (preferably type 2 diabetes) patient, wherein linagliptin effects the treatment as follows:
i) without increasing the risk of (one or more) three point major adverse cardiovascular events (3P-MACE), wherein the one or more three point major adverse cardiovascular events (3P-MACE) are selected from the group consisting of cardiovascular death, nonfatal myocardial infarction (MI) and nonfatal stroke,
ii) without increasing the risk of hospitalization for heart failure,
iii) without increasing the risk of all-cause mortality,
iv) without increasing the risk of cardiovascular (CV) death,
v) without increasing the risk of (one or more) renal outcome events, wherein the one or more renal outcome events are selected from the group consisting of renal death, sustained end stage renal disease (ESRD) and sustained decrease of 40% or more in estimated glomerular filtration rate (eGFR),
vi) with preventing, delaying the occurrence or reducing the risk of albuminuria progression, wherein the albuminuria progression is selected from the group consisting of change from normal albuminuria to micro-or macroalbuminuria and change from microalbuminuria to macroalbuminuria, and/or
vii) with preventing, delaying the occurrence or reducing the risk of (one or more) microvascular renal and/or eye complications, wherein the one or more microvascular renal and/or eye complications are selected from the group consisting of renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and diabetes-related-blindness.

The present invention relates to linagliptin, optionally in combination with one or more other active agents, for use in a method of treating a diabetic (preferably type 2 diabetes) patient (particularly without increasing the risk of cardiovascular and/or renal complications or events), said method comprising administering linagliptin, optionally in combination with one or more other active agents, to the patient in need thereof,
wherein treatment of said patient with linagliptin does not increase the rate of (primary cardiovascular, 3P-MACE) composite endpoint of cardiovascular death, nonfatal myocardial infarction (MI) or nonfatal stroke compared to a patient treated with placebo, and/or
wherein treatment of said patient with linagliptin does not increase the rate of hospitalization for heart failure compared to a patient treated with placebo, and/or
wherein treatment of said patient with linagliptin does not increase the rate of all-cause mortality compared to a patient treated with placebo, and/or
wherein treatment of said patient with linagliptin does not increase the rate of cardiovascular death compared to a patient treated with placebo, and/or
wherein treatment of said patient with linagliptin does not increase the rate of (secondary renal) composite endpoint of renal death, sustained end stage renal disease (ESRD) or sustained decrease of 40% or more in estimated glomerular filtration rate (eGFR) compared to a patient treated with placebo, and/or
wherein treatment of said patient with linagliptin does not increase the rate of (albuminuria progression) composite endpoint of change from normal albuminuria to micro-or macroalbuminuria or change from microalbuminuria to macroalbuminuria compared to a patient treated with placebo, and/or
wherein treatment of said patient with linagliptin does not increase the rate of composite (microvascular, renal and eye outcomes) endpoint of renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage or diabetes-related-blindness compared to a patient treated with placebo.

In certain instances, the present invention relates to linagliptin, optionally in combination with one or more other active agents, for use in the treatment of diabetes (preferably type 2 diabetes) in a patient in need thereof, wherein the treatment is characterized in that linagliptin reduces the risk of, prevents or delays (the time to first) occurrence of hospitalization for heart failure.

In an embodiment, the present invention relates to linagliptin, optionally in combination with one or more other active agents (which do not include an insulin), for use in the treatment of diabetes (preferably type 2 diabetes) in a patient in need thereof, wherein the treatment is characterized in that linagliptin reduces the risk of, prevents or delays (the time to first) occurrence of hospitalization for heart failure, wherein the patient is not on background medication with an insulin.

In certain instances, the present invention relates to linagliptin, optionally in combination with one or more other active agents, for use in the treatment of diabetes (preferably type 2 diabetes) in a patient in need thereof, wherein the treatment is characterized in that linagliptin reduces the risk of, prevents, slows or delays (the time to first) occurrence of albuminuria progression, wherein the albuminuria progression includes change from normal albuminuria to micro-or macroalbuminuria and/or change from microalbuminuria to macroalbuminuria.

In an embodiment, said risk of albuminuria progression is reduced by the treatment from about 10% to about 20% compared to placebo, such as reduced about 14% compared to placebo.

In certain instances, the present invention relates to linagliptin, optionally in combination with one or more other active agents, for use in the treatment of diabetes (preferably type 2 diabetes) in a patient in need thereof, wherein the treatment is characterized in that linagliptin reduces the risk of, prevents or delays (the time to first) occurrence of microvascular renal and/or eye complications, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness.

In an embodiment, said risk of microvascular renal and/or eye complications is reduced by the treatment from about 10% to about 20% compared to placebo, such as reduced about 14% compared to placebo.

In a particular embodiment, the patient according to the present invention is a subject having diabetes (e.g. type 1 or type 2 diabetes or LADA, particularly type 2 diabetes).

In particular, the patient according to the present invention is a human, particularly, a human adult.

Especially, the patient according to the present invention is a human type 2 diabetes patient.

The diabetes (preferably type 2 diabetes) patients according to the present invention include patients with high or increased cardiovascular (CV) and/or renal risk, such as e.g. evidenced by a history of established macrovascular and/or renal disease (e.g. as defined herein), such as e.g. wherein the diabetes patient has evidence of prevalent kidney disease or compromised kidney function, with or without macrovascular (cardiovascular) disease, such as defined by i) albuminuria and previous macrovascular disease and/or ii) impaired renal function with predefined urine albumin creatinine ratio (UACR).

In a special embodiment, the diabetes patients according to the present invention include patients who have (had) or are at-risk of (micro-and/or macro-) vascular diseases, complications or events, e.g. such patients are at high vascular risk, especially at high risk of both CV and kidney complications or (major) events, particularly such patients have evidence of compromised kidney function with or without CV disease.

For example, such patients according to the present invention at high vascular risk have (Condition a):
both
albuminuria (e.g. micro-or macro-albuminuria)
and
previous macrovascular (e.g. cardio-or cerebrovascular) disease (such as e.g. myocardial infarction, coronary artery disease, (ischemic or haemorrhagic) stroke, carotid artery disease and/or peripheral artery disease);
and/or
either
(mild or moderate) renal impairment (e.g. CKD stage 1, 2 or 3, such as CKD stage 1, 2 (mild) or 3a (mild-moderate), preferably eGFR ≥45-75 mL/min/1.73 m²) with macro-albuminuria,
or
(moderate or severe) renal impairment (e.g. CKD stage 3 or 4, such as CKD stage 3b (moderate-severe) or 4 (severe), preferably eGFR 15-45 mL/min/1.73 m²), with or without any albuminuria (such as e.g. with or without micro- or macro-albuminuria).

In more detail, such a patient according to the present invention at high vascular risk is a patient (preferably diabetic, particularly type 2 diabetes patients) having (Condition b):
(i) albuminuria (micro or macro) (such as e.g. urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours)) and previous macrovascular disease, such as e.g. defined as one or more of a) to f):
a) previous myocardial infarction,
b) advanced coronary artery disease,
c) high-risk single-vessel coronary artery disease,
d) previous ischemic or hemorrhagic stroke,
e) presence of carotid artery disease,
f) presence of peripheral artery disease; and/or
(ii) impaired renal function (e.g. with or without CV co-morbidities), such as e.g. defined by:
impaired renal function (e.g. as defined by MDRD formula) with an estimated glomerular filtration rate (eGFR) 15-45 mL/min/1.73 m² with any urine albumin creatinine ratio (UACR), or
impaired renal function (e.g. as defined by MDRD formula) with an estimated glomerular filtration rate (eGFR) ≥45-75 mL/min/1.73 m² with an urine albumin creatinine ratio (UACR)>200 mg/g creatinine or>200 mg/l (milligram albumin per liter of urine) or >200 μg/min (microgram albumin per minute) or>200 mg/24 h (milligram albumin per 24 hours).

In further more detail, such a patient according to the present invention at high vascular risk is a patient (preferably diabetic, particularly type 2 diabetes patients) with the Condition I (embodiment 1) and/or with the Condition II (embodiment 2), each as defined hereinbelow.

### Condition I:

albuminuria (such as e.g. urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours)) and
previous macrovascular disease, such as e.g. defined as one or more of a) to f):
a) previous myocardial infarction (e.g. ≥2 months),
b) advanced coronary artery disease, such as e.g. defined by any one of the following:
≥50% narrowing of the luminal diameter in 2 or more major coronary arteries (e.g. LAD, CX or RCA) by coronary angiography or CT angiography,
left main stem coronary artery with ≥50% narrowing of the luminal diameter,
prior percutaneous or surgical revascularization of ≥2 major coronary arteries (e.g. ≥2 months),
combination of prior percutaneous or surgical revascularization, such as e.g. of 1 major coronary artery (e.g. ≥2 months) and ≥50% narrowing of the luminal diameter by coronary angiography or CT angiography of at least 1 additional major coronary artery,
c) high-risk single-vessel coronary artery disease, such as e.g. defined as the presence of ≥50% narrowing of the luminal diameter of one major coronary artery (e.g. by coronary angiography or CT angiography in patients not revascularised) and at least one of the following:
a positive non invasive stress test, such as e.g. confirmed by either:
a positive ECG exercise tolerance test in patients without left bundle branch block, Wolff-Parkinson-White syndrome, left ventricular hypertrophy with repolarization abnormality, or paced ventricular rhythm, atrial fibrillation in case of abnormal ST-T segments,
a positive stress echocardiogram showing induced regional systolic wall motion abnormalities,
a positive nuclear myocardial perfusion imaging stress test showing stress induced reversible perfusion abnormality,
patient discharged from hospital with a documented diagnosis of unstable angina pectoris (e.g. ≥2-12 months),
d) previous ischemic or haemorrhagic stroke (e.g. >3 months),
e) presence of carotid artery disease (e.g. symptomatic or not), such as e.g. documented by either:
imaging techniques with at least one lesion estimated to be ≥50% narrowing of the luminal diameter,
prior percutaneous or surgical carotid revascularization,
f) presence of peripheral artery disease, such as e.g. documented by either:
previous limb angioplasty, stenting or bypass surgery,
previous limb or foot amputation due to macrocirculatory insufficiency,
angiographic evidence of peripheral artery stenosis ≥50% narrowing of the luminal diameter in at least one limb (e.g. definition of peripheral artery: common iliac artery, internal iliac artery, external iliac artery, femoral artery, popliteal artery),

### Condition II:

impaired renal function (e.g. with or without CV co-morbidities), such as e.g. defined by:
impaired renal function (e.g. as defined by MDRD formula) with an eGFR 15-45 mL/min/1.73 m² with any urine albumin creatinine ratio (UACR), or
impaired renal function (e.g. as defined by MDRD formula) with an eGFR ≥45-75 mL/min/1.73 m² with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24 hours).

In a further embodiment, patients according to the present invention include, without being limited to, patients with long standing type 2 diabetes, e.g. with duration of type 2 diabetes mellitus of >5 years or >10 years or >15 years.

In a further embodiment, patients according to the present invention include, without being limited to, elderly patients, e.g. >65 years of age or ≥75 years of age.

In a further embodiment, patients according to the present invention include, without being limited to, patients with renal impairment.

In a further embodiment, patients according to the present invention include, without being limited to, patients with mild renal impairment (eGFR ≥60 to <90 mL/min/1.73 m²).

In a further embodiment, patients according to the present invention include, without being limited to, patients with moderate renal impairment (eGFR ≥45 to <60 mL/min/1.73 m²).

In a further embodiment, patients according to the present invention include, without being limited to, patients with moderately severe renal impairment (eGFR ≥30 to <45 mL/min/1.73m²).

In a further embodiment, patients according to the present invention include, without being limited to, patients with severe renal impairment (eGFR <30 mL/min/1.73 m²).

In a further embodiment, patients according to the present invention include, without being limited to, patients with normal renal function (eGFR ≥90 mL/min/1.73 m²).

In a further embodiment, patients according to the present invention include, without being limited to, patients with microalbuminuria (UACR 30-300 mg/g).

In a further embodiment, patients according to the present invention include, without being limited to, patients with macroalbuminuria (UACR >300 mg/g).

In a further embodiment, patients according to the present invention include, without being limited to, patients with normal abuminuria (UACR <30 mg/g).

In a further embodiment, patients according to the present invention include, without being limited to, patients with kidney disease such as e.g.
having i) albuminuria, such as e.g. microalbuminuria (UACR 30-300 mg/g) or macroalbuminuria (UACR >300 mg/g), and/or
having ii) impaired renal function, such as e.g. mild (eGFR ≥60 to <90 mL/min/1.73 m²), moderate (eGFR ≥45 to <60 mL/min/1.73 m²), moderate/severe (eGFR ≥30 to <45 mL/min/1.73 m²) or severe (eGFR <30 mL/min/1.73 m²) renal impairment;
in a particular sub-embodiment, patients according to the present invention have both albuminuria and renal impairment.

In a further embodiment, patients according to the present invention include, without being limited to, patients with one or two antidiabetic background medications.

In a further embodiment, patients according to the present invention include, without being limited to, patients with at least one antidiabetic background medication, which includes metformin.

In a further embodiment, patients according to the present invention include, without being limited to, patients with at least one antidiabetic background medication, which includes a sulfonylurea.

In a further embodiment, patients according to the present invention include, without being limited to, patients with at least one antidiabetic background medication, which includes an insulin.

In a further embodiment, patients according to the present invention include, without being limited to, patients with at least one antidiabetic background medication, which does not include an insulin.

In a further embodiment, patients according to the present invention include, without being limited to, patients with at least one background medication to reduce cardiovascular risk.

In a further embodiment, patients according to the present invention include, without being limited to, patients with at least one background medication to reduce cardiovascular risk, which is aspirin or a platelet aggregation inhibitor.

In a further embodiment, patients according to the present invention include, without being limited to, patients with at least one background medication to reduce cardiovascular risk, which is a statin.

In a further embodiment, patients according to the present invention include, without being limited to, patients with at least one background medication to reduce cardiovascular risk, which is an ACE (angiotensin converting enzyme) inhibitor or an ARB (angiotensin receptor blocker).

In a further embodiment, patients according to the present invention include, without being limited to, patients with at least one background medication to reduce cardiovascular risk, which is an ACE inhibitor, an ARB, a beta blocker, a diuretic or a calcium channel blocker.

In a further embodiment, patients according to the present invention include, without being limited to, patients who are overweight.

In a further embodiment, patients according to the present invention include, without being limited to, patients who are obese.

In a further embodiment, patients according to the present invention include, without being limited to, patients who are of normal weight.

In a further embodiment, patients according to the present invention include, without being limited to, patients who are from Europe region.

In a further embodiment, patients according to the present invention include, without being limited to, patients who are from North America region.

In a further embodiment, patients according to the present invention include, without being limited to, patients who are from South America region.

In a further embodiment, patients according to the present invention include, without being limited to, patients who are from Asia region.

In a further embodiment, patients according to the present invention include, without being limited to, patients at high risk for adverse kidney events (prognosis of CKD by eGFR and albuminuria categories):

### High Risk:

UACR (mg/g) >300 and eGFR (ml/min/1.73 m²) >60, or
UACR (mg/g) 30-299 and eGFR (ml/min/1.73 m²) 45-59, or
UACR (mg/g) <30 and eGFR (ml/min/1.73 m²) 30-44.

In a further embodiment, patients according to the present invention include, without being limited to, patients at very high risk for adverse kidney events (prognosis of CKD by eGFR and albuminuria categories):

### Very High Risk:

UACR (mg/g) >300 and eGFR (ml/min/1.73 m²) 45-59 or 30-44 or <30, or
UACR (mg/g) 30-299 and eGFR (ml/min/1.73 m²) 30-44 or <30, or
UACR (mg/g) <30 and eGFR (ml/min/1.73 m²) <30.

Accordingly, the present invention relates to linagliptin, optionally in combination with one or more other active agents, for use in the treatment of diabetes (preferably type 2 diabetes) patients with or at-risk of (micro-and/or macro-) vascular diseases, such as e.g. patients having or being at-risk of cardiovascular and/or microvascular (e.g. renal) diseases, such as e.g. patients at high or increased vascular (cardio-renal) risk (such as e.g. described hereinabove and hereinbelow, e.g. having Condition a, Condition b, Condition I, or Condition II),

In an embodiment, the present invention relates to linagliptin, optionally in combination with one or more other active agents, for use in the treatment of diabetes (preferably type 2 diabetes) patients characterized in that the patients are male or female patients who before commencement of treatment with linagliptin
are drug-naïve or pre-treated with any antidiabetic background medication, excluding treatment with GLP-1 receptor agonists, DPP-4 inhibitors or SGLT-2 inhibitors for 7 or more consecutive days,
receive antidiabetic background medication with an unchanged daily dose for at least 8 weeks, wherein if insulin is part of the background therapy, the average daily insulin dose should not have changed by more than 10% within the 8 weeks compared with the daily insulin dose at commencement,
have an HbA1c of ≥6.5% and ≤10.0%,
have a Body Mass Index (BMI) ≤45 kg/m², and
have a high risk of cardiovascular or renal events defined by a) albuminuria and previous macrovascular disease and/or b) impaired renal function with predefined UACR, such as e.g.
(i) albuminuria (micro or macro) (such as e.g. urine albumin creatinine ratio (UACR) ≥ 30 mg/g creatinine or ≥30 mg/l (milligram albumin per liter of urine) or ≥30 μg/min (microgram albumin per minute) or ≥30 mg/24 h (milligram albumin per 24 hours)) and
   previous macrovascular disease, such as e.g. defined as one or more of a) to f):
   a) previous myocardial infarction,
   b) advanced coronary artery disease,
   c) high-risk single-vessel coronary artery disease,
   d) previous ischemic or haemorrhagic stroke,
   e) presence of carotid artery disease,
   f) presence of peripheral artery disease; and/or
   (ii) impaired renal function (e.g. with or without CV co-morbidities), such as e.g. defined by:
   impaired renal function (e.g. as defined by MDRD formula) with an estimated glomerular filtration rate (eGFR) 15-45 mL/min/1.73 m² with any urine albumin creatinine ratio (UACR), or
   impaired renal function (e.g. as defined by MDRD formula) with an estimated glomerular filtration rate (eGFR) ≥45-75 mL/min/1.73 m² with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24 hours).

Also, the present invention relates to a method of treating a diabetic (preferably type 2 diabetes) patient with increased or high vascular risk (e.g. increased risk of (micro-and/or macro-) vascular diseases, such as increased cardiovascular and/or renal risk) based on established macrovascular disease and/or microvascular (renal) disease (such as e.g. defined herein by a) albuminuria and previous macrovascular disease and/or b) impaired renal function with predefined UACR), e.g. cf. Condition a, Condition b, Condition I, or Condition II), the method comprising treating the patient with linagliptin (optionally in combination with one or more other active agents).

Further, the present invention relates to a method of treating a diabetic (preferably type 2 diabetes) patient at increased or high vascular risk (e.g. at increased of (micro-and/or macro-) vascular diseases, such as increased cardiovascular and/or renal risk) based on established macrovascular disease and/or microvascular (renal) disease (such as e.g. described herein, e.g. having Condition a, Condition b, Condition I, or Condition II),
i) without increasing the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke,
ii) without increasing the risk of hospitalization for heart failure,
iii) without increasing the risk of key renal outcome events, wherein the key renal outcome events include renal death, sustained end stage renal disease (ESRD) and/or sustained decrease of 40% or more in estimated glomerular filtration rate (eGFR),
iv) with preventing or reducing the risk of albuminuria progression, wherein the albuminuria progression includes change from normal albuminuria to micro-or macroalbuminuria and/or change from microalbuminuria to macroalbuminuria, and/or
v) with preventing or reducing the risk of microvascular renal and/or eye complications, wherein the microvascular renal and/or eye complications include renal death, sustained ESRD,
sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness;
the method comprising treating the patient with linagliptin (optionally in combination with one or more other active agents).

In certain embodiments, such treatment of a patient with or at-risk of (micro-and/or macro-) vascular diseases may further comprise the step of identifying such a patient, such as e.g. based on established macrovascular disease and/or microvascular (renal) disease such as described herein (such as e.g. based on evidence of compromised kidney function with or without CV disease, such as described herein, e.g. cf. Condition a, Condition b, Condition I, or Condition II), prior to treatment with linagliptin.

Yet accordingly, the present invention relates to linagliptin, optionally in combination with one or more other active agents, for use in the treatment of diabetes (preferably type 2 diabetes) in patients in need thereof, wherein the treatment is characterized in that:
i) linagliptin does not increase the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke,
ii) linagliptin does not increase the risk of hospitalization for heart failure,
iii) linagliptin does not increase the risk of key renal outcome events, wherein the key renal outcome events include renal death, sustained end stage renal disease (ESRD) and/or sustained decrease of 40% or more in estimated glomerular filtration rate (eGFR),
iv) linagliptin prevents or reduces the risk of albuminuria progression, wherein the albuminuria progression includes change from normal albuminuria to micro-or macroalbuminuria and/or change from microalbuminuria to macroalbuminuria, and/or
v) linagliptin prevents or reduces the risk of microvascular renal and/or eye complications, wherein the microvascular renal and/or eye complications include renal death, sustained ESRD,
sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness;
including in (human) patients with or at-risk of (micro-and/or macro-) vascular diseases, such as e.g. patients having or being at-risk of cardiovascular and/or microvascular (e.g. renal) diseases, such as e.g. patients at high or increased vascular (cardio-renal) risk (such as e.g. described hereinabove and hereinbelow, e.g. having Condition a, Condition b, Condition I, or Condition II).

In an embodiment, the present invention relates to linagliptin, optionally in combination with one or more other active agents, for use in the treatment of a diabetic (preferably type 2 diabetes) patient at risk of heart failure, wherein linagliptin effects the treatment without increasing the risk of hospitalization for heart failure.

In a further embodiment, the present invention relates to a method of treating a diabetic (preferably type 2 diabetes) patient at risk of heart failure without increasing the risk of hospitalization for heart failure, the method comprising treating the patient with linagliptin (optionally in combination with one or more other active agents).

Such treatment of a patients with risk of heart failure may further comprise the step of identifying such patient, such as e.g. based on established macrovascular disease and/or microvascular (renal) disease such as described herein (such as e.g. based on evidence of compromised kidney function with or without CV disease, such as described herein, e.g. having Condition a, Condition b, Condition I, or Condition II), prior to treatment with linagliptin.

Still yet accordingly, the present invention relates to linagliptin, optionally in combination with one or more other active agents, for use in the treatment of diabetic (preferably type 2 diabetes) patients,
i) wherein linagliptin effects the treatment without increasing the risk of 3 point major adverse cardiovascular events (3P-MACE), wherein the 3 point major adverse cardiovascular events (3P-MACE) include cardiovascular death, nonfatal myocardial infarction (MI) and/or nonfatal stroke,
ii) wherein linagliptin effects the treatment without increasing the risk of hospitalization for heart failure,
iii) wherein linagliptin effects the treatment without increasing the risk of key renal outcome events, wherein the key renal outcome events include renal death, sustained end stage renal disease (ESRD) and/or sustained decrease of 40% or more in estimated glomerular filtration rate (eGFR),
iv) wherein linagliptin effects the treatment with preventing or reducing the risk of albuminuria progression, wherein the albuminuria progression includes change from normal albuminuria to micro-or macroalbuminuria and/or change from microalbuminuria to macroalbuminuria, and/or
v) wherein linagliptin effects the treatment with preventing or reducing the risk of microvascular renal and/or eye complications, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness;
including in (human) patients at high or increased vascular (cardio-renal) risk (such as at high or increased risk of cardiovascular and/or renal events), such as based on (history of) established macrovascular disease and/or renal disease (e.g. albuminuria and/or impaired renal function), such as defined by i) albuminuria and previous macrovascular disease and/or ii) impaired renal function with predefined urine albumin creatinine ratio (UACR), such as having:
(i) albuminuria (micro or macro) (such as e.g. urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours)) and previous macrovascular disease, such as e.g. defined as one or more of a) to f):
a) previous myocardial infarction,
b) advanced coronary artery disease,
c) high-risk single-vessel coronary artery disease,
d) previous ischemic or haemorrhagic stroke,
e) presence of carotid artery disease,
f) presence of peripheral artery disease;
and/or
(ii) impaired renal function (e.g. with or without CV co-morbidities), such as e.g. defined by:
impaired renal function (e.g. as defined by MDRD formula) with an estimated glomerular filtration rate (eGFR) 15-45 mL/min/1.73 m² with any urine albumin creatinine ratio (UACR), or
impaired renal function (e.g. as defined by MDRD formula) with an estimated glomerular filtration rate (eGFR) ≥45-75 mL/min/1.73 m² with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24 hours)).

In certain instances, the present invention relates to linagliptin, optionally in combination with one or more other active agents, for use in the treatment of diabetic (preferably type 2 diabetes) patients wherein linagliptin effects the treatment with reducing the risk, preventing, protecting against, delaying the occurrence of, delaying the progression of and/or treating a micro-(renal or eye) or macrovascular (cardio-or cerebrovascular) disease, complication or event; including in (human) patients with or at-risk of (micro-and/or macro-) vascular diseases, such as e.g. patients having or being at-risk of cardiovascular and/or microvascular (e.g. renal) diseases, such as e.g. patients at high or increased vascular (cardio-renal) risk (such as e.g. described hereinabove and hereinbelow, e.g. having Condition a, Condition b, Condition I, or Condition II).

Within the meaning or purpose of the present application, any risk features/properties of linagliptin may be relative to placebo. Any (risk) analysis of data may be based on the hazard ratio (HR) (and its statistically significance) such as found in a drug study using linagliptin compared to placebo (on top of standard of care). Alternatively, any analysis of data may be based on numerical differences (e.g. number of incidences, such as e.g. without reaching statistical significance) such as found in a drug study using linagliptin compared to placebo (on top of standard of care).

Duration of treatment with linagliptin (preferably 5 mg per day, administered orally, optionally in combination with one or more other active substances, e.g. such as those described herein) for the purpose of the present invention may be over a lengthy period, such as e.g. at least 1-5 years, or at least 12-48 months, or at least 18-54 months, preferably at least about 20-24 months. In an embodiment, the median treatment exposure is at least about 1.8 or 1.9 years. In an embodiment, the patients are followed for at least 2.2 years.

Other aspects of the present invention become apparent to the skilled person from the foregoing and following remarks (including the examples and claims).

### DETAILED DESCRIPTION OF THE INVENTION

A particularly preferred DPP-4 inhibitor to be emphasized within the present invention is linagliptin. The term “linagliptin” as employed herein refers to linagliptin or a pharmaceutically acceptable salt thereof, including hydrates and solvates thereof, and amorphous or crystalline forms thereof, preferably linagliptin refers to 1-[(4-methyl-quinazolin-2-yl) methyl]-3-methyl-7-(2-butyn-1-yl)-8-(3-(R)-amino-piperidin-1-yl)-xanthine.

Preferably, linagliptin is administered in an oral daily dose of 5 mg (e.g. 2.5 mg twice daily, or—preferably—5 mg once daily).

### Further Embodiments

In an embodiment, diabetes patients as referred to herein may include patients who have not previously been treated with an antidiabetic drug (drug-naïve patients). Thus, in an embodiment, the treatments described herein may be used in naïve patients. In certain embodiments of the treatments of this invention, the DPP-4 inhibitor (preferably linagliptin) may be used alone or in combination with one or more other antidiabetics in such patients. In another embodiment, diabetes patients within the meaning of this invention may include patients pre-treated with conventional antidiabetic background medication, such as e.g. patients with advanced or late stage type 2 diabetes mellitus (including patients with failure to conventional antidiabetic therapy), such as e.g. patients with inadequate glycemic control on one, two or more conventional oral and/or non-oral antidiabetic drugs as defined herein, such as e.g. patients with insufficient glycemic control despite (mono-) therapy with metformin, a thiazolidinedione (particularly pioglitazone), a sulphonylurea, a glinide, GLP-1 or GLP-1 analogue, insulin or insulin analogue, or an α-glucosidase inhibitor, or despite dual combination therapy with metformin/sulphonylurea, metformin/thiazolidinedione (particularly pioglitazone), sulphonylurea/α-glucosidase inhibitor, pioglitazone/sulphonylurea, metformin/insulin, pioglitazone/insulin or sulphonylurea/insulin. Thus, in an embodiment, the treatments described herein may be used in patients experienced with therapy, e.g. with conventional oral and/or non-oral antidiabetic mono-or dual or triple combination medication as mentioned herein. In certain embodiments of the therapies of this invention, in such patients the DPP-4 inhibitor (preferably linagliptin) may be used on top of or added on the existing or ongoing conventional oral and/or non-oral antidiabetic mono- or dual or triple combination medication with which such patients are pre-treated or experienced.

For example, a diabetes patient (particularly type 2 diabetes patient, with insufficient glycemic control) as referred to herein may be treatment-naïve or pre-treated with one or more (e.g. one or two) conventional antidiabetic agents selected from metformin, thiazolidinediones (particularly pioglitazone), sulphonylureas, glinides, α-glucosidase inhibitors (e.g. acarbose, voglibose), and insulin or insulin analogues, such as e.g. pre-treated or experienced with:
metformin, α-glucosidase inhibitor, sulphonylurea or glinide monotherapy, or metformin plus α-glucosidase inhibitor, metformin plus sulphonylurea, metformin plus glinide, α-glucosidase inhibitor plus sulphonylurea, or α-glucosidase inhibitor plus glinide dual combination therapy.

In certain embodiments relating to such treatment-naïve patients, the DPP-4 inhibitor (preferably linagliptin) may be used as monotherapy, or as initial combination therapy such as e.g. with metformin, a thiazolidinedione (particularly pioglitazone), a sulphonylurea, a glinide, an α-glucosidase inhibitor (e.g. acarbose, voglibose), GLP-1 or GLP-1 analogue, or insulin or insulin analogue; preferably as monotherapy.

In certain embodiments relating to such patients pre-treated or experienced with one or two conventional antidiabetic agents, the DPP-4 inhibitor (preferably linagliptin) may be used as as add-on combination therapy, i.e. added to an existing or background therapy with the one or two conventional antidiabetics in patients with insufficient glycemic control despite therapy with the one or more conventional antidiabetic agents, such as e.g. as add-on therapy to one or more (e.g. one or two) conventional antidiabetics selected from metformin, thiazolidinediones (particularly pioglitazone), sulphonylureas, glinides, α-glucosidase inhibitors (e.g. acarbose, voglibose), GLP-1 or GLP-1 analogues, and insulin or insulin analogues, such as e.g.: as add-on therapy to metformin, to a α-glucosidase inhibitor, to a sulphonylurea or to a glinide;
or as add-on therapy to metformin plus α-glucosidase inhibitor, to metformin plus sulphonylurea, to metformin plus glinide, to α-glucosidase inhibitor plus sulphonylurea, or to α-glucosidase inhibitor plus glinide;
or as add-on therapy to an insulin, with or without metformin, a thiazolidinedione (particularly pioglitazone), a sulphonylurea, a glinide or an α-glucosidase inhibitor (e.g. acarbose, voglibose).

A further embodiment of diabetic patients as described herein may relate to patients ineligible for metformin therapy including
patients for whom metformin therapy is contraindicated, e.g. patients having one or more contraindications against metformin therapy according to label, such as for example patients with at least one contraindication selected from:
   renal disease, renal impairment or renal dysfunction (e.g., as specified by product information of locally approved metformin),
   dehydration,
   unstable or acute congestive heart failure,
   acute or chronic metabolic acidosis, and
   hereditary galactose intolerance;
and
patients who suffer from one or more intolerable side effects attributed to metformin, particularly gastrointestinal side effects associated with metformin, such as for example patients suffering from at least one gastrointestinal side effect selected from:
   nausea,
   vomiting,
   diarrhoea,
   intestinal gas, and
   severe abdominal discomfort.

A further embodiment of diabetes patients as referred to herein may include, without being limited to, those diabetes patients for whom normal metformin therapy is not appropriate, such as e.g. those diabetes patients who need reduced dose metformin therapy due to reduced tolerability, intolerability or contraindication against metformin or due to (mildly) impaired/reduced renal function (including elderly patients, such as e.g. ≥60-65 years).

A further embodiment of diabetes patients may refer to patients having renal disease, renal dysfunction, or insufficiency or impairment of renal function (including mild, moderate and/or severe renal impairment), e.g. as may be suggested (if not otherwise noted) by elevated serum creatinine levels (e.g. serum creatinine levels above the upper limit of normal for their age, e.g. ≥130-150 μmol/l, or ≥1.5 mg/dl (≥136 μmol/l) in men and ≥1.4 mg/dl (≥124 μmol/l) in women) or abnormal creatinine clearance (e.g. glomerular filtration rate (GFR) ≤30-60 ml/min).

In this context, in a further embodiment, mild renal impairment may be e.g. suggested (if not otherwise noted) by a creatinine clearance of 50-80 ml/min (approximately corresponding to serum creatine levels of ≤1.7 mg/dL in men and ≤1.5 mg/dl in women); moderate renal impairment may be e.g. suggested (if not otherwise noted) by a creatinine clearance of 30-50ml/min (approximately corresponding to serum creatinine levels of >1.7 to ≤3.0 mg/dl in men and >1.5 to ≤2.5 mg/dl in women); and severe renal impairment may be e.g. suggested (if not otherwise noted) by a creatinine clearance of<30 ml/min (approximately corresponding to serum creatinine levels of >3.0 mg/dl in men and >2.5 mg/dl in women). Patients with end-stage renal disease require dialysis (e.g. hemodialysis or peritoneal dialysis).

In another further embodiment, patients with renal disease, renal dysfunction or renal impairment may include patients with chronic renal insufficiency or impairment, which can be stratified (if not otherwise noted) according to glomerular filtration rate (GFR, ml/min/1.73m²) into 5 disease stages: stage 1 characterized by normal GFR ≥90 (optionally plus either persistent albuminuria (e.g. UACR ≥30 mg/g) or known structural or hereditary renal disease); stage 2 characterized by mild reduction of GFR (GFR 60-89) describing mild renal impairment; stage 3 characterized by moderate reduction of GFR (GFR 30-59) describing moderate renal impairment [or in more detail: stage 3a characterized by mild-moderate reduction of GFR (GFR 45-59) describing mild-moderate renal impairment, stage 3b characterized by moderate-severe reduction of GFR (GFR 30-44) describing moderate-severe renal impairment]; stage 4 characterized by severe reduction of GFR (GFR 15-29) describing severe renal impairment; and terminal stage 5 characterized by requiring dialysis or GFR<15 describing established kidney failure (end-stage renal disease, ESRD).

Chronic kidney disease and its stages (CKD 1-5) can be usually characterized or classified accordingly, such as based on the presence of either kidney damage (albuminuria) or impaired estimated glomerular filtration rate (GFR <60 [ml/min/1.73m²], with or without kidney damage).

Albuminuria stages may be for example classified as disclosed herein and/or by urine albumin creatinine ratio (such as usually UACR ≥30 mg/g, in some instances ≥20 μg/min albumin excretion rate), such as e.g. microalbuminuria may be for example classified by UACR 30-300mg/g (in some instances 20-200 g/min) or, in another embodiment, by UACR 30-200 mg/g, and/or macroalbuminuria may be for example classified by UACR >300 mg/g (in some instances >200 μg/min), or, in another embodiment, by UACR >200 mg/g. Very high UACR ≥2000 mg/g may be classified as nephrotic.

A further embodiment of diabetic patients may refer to patients with inadequate control of albuminuria despite therapy with an angiotensin-converting enzyme (ACE) inhibitor and/or an angiotensin II receptor blocker (ARB).

A further embodiment of diabetic patients may refer to patients (preferably diabetic patients, particularly type 2 diabetes patients) having micro-(renal-) and/or macro-(cardiovascular-) disease history and/or medications, such as CKD/diabetic nephropathy, renal impairment and/or (micro-or macro) albuminuria, and/or macrovascular disease (e.g. coronary artery disease, peripheral artery disease, cerebrovascular disease, hypertension), and/or microvascular disease (e.g. diabetic nephropathy, neuropathy, retinopathy), and/or on acetylsalicylic acid, antihypertensive and/or lipid lowering medication, such as e.g. on (previous or ongoing) therapy with acetylsalicylic acid, an ACE inhibitor, ARB, beta-blocker, Calcium-antagonist or diuretic, or combination thereof, and/or on (previous or ongoing) therapy with a fibrate, niacin or statin, or combination thereof.

A further embodiment of diabetic patients may refer to patients with diabetic nephropathy (with or without additional standard background therapy such as e.g. with an ACEi or ARB), e.g. including a vulnerable diabetic nephropathy patient such as who are aged ≥65 years typically having longer diabetes duration (>5 years), renal impairment (such as mild (60 to <90 eGFR ml/min/1.73 m²) or moderate (30 to <60 eGFR ml/min/1.73 m²) renal impairment) and/or higher baseline UACR (such as advanced stages of micro- or macroalbuminuria).

A further embodiment of diabetic patients may refer to patients with diabetic nephropathy, especially in those patients on (e.g. previous or ongoing) therapy with an angiotensin-converting enzyme (ACE) inhibitor and/or an angiotensin II receptor blocker (ARB), such as e.g. patients with inadequate control of albuminuria despite therapy with an angiotensin-converting enzyme (ACE) inhibitor and/or an angiotensin II receptor blocker (ARB).

The DPP-4 inhibitor may be administered in combination (e.g. on-top, add-on) with the background medication such as e.g. angiotensin-converting enzyme (ACE) inhibitor or the angiotensin II receptor blocker (ARB), to the patient.

Within this invention it is to be understood that combinations, compositions or combined uses according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The combined administration of this invention may take place by administering the active components or ingredients together, such as e.g. by administering them simultaneously in one single or in two separate formulations or dosage forms. Alternatively, the administration may take place by administering the active components or ingredients sequentially, such as e.g. successively in two separate formulations or dosage forms.

For the combination therapy of this invention the active components or ingredients may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination.

Unless otherwise noted, combination therapy may refer to first line, second line or third line therapy, or initial or add-on combination therapy or replacement therapy.

Unless otherwise noted, monotherapy may refer to first line therapy (e.g. therapy of patients with insufficient glycemic control by diet and exercise alone, such as e.g. drug-naive patients, typically patients early after diagnosis and/or who have not been previously treated with an antidiabetic agent, and/or patients ineligible for metformin therapy such as e.g. patients for whom metformin therapy is contraindicated, such as e.g. due to renal impairment, or inappropriate, such as e.g. due to intolerance).

Unless otherwise noted, add-on combination therapy may refer to second line or third line therapy (e.g. therapy of patients with insufficient glycemic control despite (diet and exercise plus) therapy with one or two conventional antidiabetic agents, typically patients who are pre-treated with one or two antidiabetic agents, such as e.g. patients with such existing antidiabetic background medication).

Unless otherwise noted, initial combination therapy may refer to first line therapy (e.g. therapy of patients with insufficient glycemic control by diet and exercise alone, such as e.g. drug-naive patients, typically patients early after diagnosis and/or who have not been previously treated with an antidiabetic agent).

As different metabolic functional disorders often occur simultaneously, it is quite often indicated to combine a number of different active principles with one another. Thus, depending on the functional disorders diagnosed, improved treatment outcomes may be obtained if a DPP-4 inhibitor is combined with one or more active substances customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The DPP-4 inhibitors mentioned above—besides their use in mono-therapy—may also be used in conjunction with one or more other active substances, by means of which improved treatment results can be obtained. Such a combined treatment may be given as a free combination of the substances or in the form of a fixed combination, for example in a tablet or capsule. Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide; nateglinide; repaglinide; mitiglinide; thiazolidinediones such as rosiglitazone and pioglitazone; alphα-glucosidase blockers such as acarbose, voglibose and miglitol; insulin and insulin analogues such as human insulin, insulin lispro, insulin glusilin, r-DNA-insulinaspart, NPH insulin, insulin detemir, insulin degludec, insulin tregopil, insulin zinc suspension and insulin glargin; amylin and amylin analogues (e.g. pramlintide or davalintide); GLP-1 and GLP-1 analogues such as Exendin-4, e.g. exenatide, exenatide LAR, liraglutide, taspoglutide, lixisenatide (AVE-0010), LY-2428757 (a PEGylated version of GLP-1), dulaglutide (LY-2189265), semaglutide or albiglutide; and/or SGLT2-inhibitors such as e.g. dapagliflozin, sergliflozin (KGT-1251), atigliflozin, canagliflozin, ipragliflozin, luseogliflozin or tofogliflozin.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

A dosage of pioglitazone is usually of about 1-10 mg, 15 mg, 30 mg, or 45 mg once a day.

Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

A dual combination of glibenclamide/metformin is usually given in doses from 1.25/250 once daily to 10/1000 mg twice daily. (typical dosage strengths are 1.25/250, 2.5/500 and 5/500 mg).

A dual combination of glipizide/metformin is usually given in doses from 2.5/250 to 10/1000 mg twice daily (typical dosage strengths are 2.5/250, 2.5/500 and 5/500 mg).

A dual combination of glimepiride/metformin is usually given in doses from 1/250 to 4/1000 mg twice daily.

A dual combination of rosiglitazone/glimepiride is usually given in doses from 4/1 once or twice daily to 4/2 mg twice daily (typical dosage strengths are 4/1, 4/2, 4/4, 8/2 and 8/4 mg).

A dual combination of pioglitazone/glimepiride is usually given in doses from 30/2 to 30/4 mg once daily (typical dosage strengths are 30/4 and 45/4 mg).

A dual combination of rosiglitazone/metformin is usually given in doses from 1/500 to 4/1000 mg twice daily (typical dosage strengths are 1/500, 2/500, 4/500, 2/1000 and 4/1000 mg).

A dual combination of pioglitazone/metformin is usually given in doses from 15/500 once or twice daily to 15/850 mg thrice daily (typical dosage strengths are 15/500 and 15/850 mg).

The non-sulphonylurea insulin secretagogue nateglinide is usually given in doses from 60 to 120mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

Acarbose is usually given in doses from 25 to 100 mg with meals. Miglitol is usually given in doses from 25 to 100 mg with meals.

Examples of combination partners that lower the lipid level in the blood are HMG-COA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists; PPAR-alpha/delta agonists; inhibitors of acyl-coenzyme A: cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); and ApoB100 antisense RNA.

A dosage of atorvastatin is usually from 1 mg to 40 mg or 10 mg to 80 mg once a day.

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin Il receptor blockers (ARBs) such as telmisartan, candesartan, valsartan, losartan, irbesartan, olmesartan, azilsartan and eprosartan.

A dosage of telmisartan is usually from 20 mg to 320 mg or 40 mg to 160 mg per day.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2inhibitors; inhibitors of tyrosine-kinases (50 mg to 600 mg) such as PDGF-receptor-kinase (cf.

EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

Further, the certain DPP-4 inhibitor of this invention may be used in combination with a substrate of DPP-4 (particularly with an anti-inflammatory substrate of DPP-4), which may be other than GLP-1, for the purposes according to the present invention, such substrates of DPP-4 include, for example—without being limited to, one or more of the following:
Incretins:
Glucagon-like peptide (GLP)-1
Glucose-dependent insulinotropic peptide (GIP)
Neuroactive:
Substance P
Neuropeptide Y (NPY)
Peptide YY
Energy homeostasis:
GLP-2
Prolactin
Pituitary adenylate cyclase activating peptide (PACAP)
Other hormones:
PACAP 27
Human chorionic gonadotrophin alpha chain
Growth hormone releasing factor (GHRF)
Luteinizing hormone alpha chain
Insulin-like growth factor (IGF-1)
CCL8/eotaxin
CCL22/macrophage-derived chemokine
CXCL9/interferon-gamma-induced monokine
Chemokines:
CXCL10/interferon-gamma-induced protein-10
CXCL11/interferon-inducible T cell a chemoattractant
CCL3L1/macrophage inflammatory protein 1 alpha isoform
LD78beta
CXCL12/stromal-derived factor 1 alpha and beta
Other:
Enkephalins, gastrin-releasing peptide, vasostatin-1,
peptide histidine methionine, thyrotropin alpha

Further or in addition, the certain DPP-4 inhibitor of this invention may be used in combination with one or more active substances which are indicated in the treatment of nephropathy, such as selected from diuretics, ACE inhibitors and/or ARBs.

Further or in addition, the certain DPP-4 inhibitor of this invention may be used in combination with one or more active substances which are indicated in the treatment or prevention of cardiovascular diseases or events (e.g. major cardiovascular events).

Moreover, optionally in addition, the certain DPP-4 inhibitor of this invention may be used in combination with one or more antiplatelet agents, such as e.g. (low-dose) aspirin (acetylsalicylic acid), a selective COX-2 or nonselective COX-1/COX-2 inhibitor, or a ADP receptor inhibitor, such as a thienopyridine (e.g. clopidogrel or prasugrel), elinogrel or ticagrelor, or a thrombin receptor antagonist such as vorapaxar.

Yet moreover, optionally in addition, the certain DPP-4 inhibitor of this invention may be used in combination with one or more anticoagulant agents, such as e.g. heparin, a coumarin (such as warfarin or phenprocoumon), a pentasaccharide inhibitor of Factor Xa (e.g. fondaparinux), or a direct thrombin inhibitor (such as e.g. dabigatran), or a Faktor Xa inhibitor (such as e.g. rivaroxaban or apixaban or edoxaban or otamixaban).

Still yet moreover, optionally in addition, the certain DPP-4 inhibitor of this invention may be used in combination with one or more agents for the treatment of heart failure (such as e.g. those mentioned in WO 2007/128761).

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

### EXAMPLES

In order that this invention be more fully understood, the herein-given examples are set forth. Further embodiments, features, effects, properties or aspects of the present invention may become apparent from the examples. The examples serve to illustrate, by way of example, the principles of the invention without restricting it.

### Cardiovascular and Renal Outcomes Trial Assessing Cardiovascular Safety and Renal Microvascular Outcome in Patients with Type 2 Diabetes at High Vascular Risk

Treatment of patients with type 2 diabetes mellitus at high cardiovascular and renal microvascular risk:

The long term impact on cardiovascular and renal (microvascular) safety, morbidity and/or mortality and relevant efficacy parameters (e.g. HbA1c, fasting plasma glucose, treatment sustainability) of treatment with linagliptin (optionally in combination with one or more other active substances, e.g. one or more other antidiabetics) in a relevant population of patients with type 2 diabetes mellitus (such as e.g. at high vascular risk and/or at advanced stage of diabetic kidney disease; such as e.g. having established CV disease, kidney disease or both) can be investigated as follows:

Type 2 diabetes patient with insufficient glycemic control (naïve or pre-treated with any antidiabetic background medication, excluding treatment with GLP-1 receptor agonists, DPP-4inhibitors or SGLT-2 inhibitors if ≥consecutive 7 days, e.g. having HbA1c 6.5-10%), and high risk of cardiovascular events, e.g. defined by:
albuminuria (micro or macro) and previous macrovascular disease: e.g. defined according to Condition I as indicated below;
and/or
impaired renal function: e.g. as defined according to Condition II as indicated below;

### Condition I:

albuminuria (such as e.g. urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours)) and previous macrovascular disease, such as e.g. defined as one or more of a) to f):
a) previous myocardial infarction (e.g. >2 months),
b) advanced coronary artery disease, such as e.g. defined by any one of the following:
≥50% narrowing of the luminal diameter in 2 or more major coronary arteries (e.g. LAD (Left Anterior Descending), CX (Circumflex) or RCA (Right Coronary Artery)) by coronary angiography or CT angiography,
left main stem coronary artery with ≥50% narrowing of the luminal diameter,
prior percutaneous or surgical revascularization of ≥2 major coronary arteries (e.g. ≥2 months),
combination of prior percutaneous or surgical revascularization, such as e.g. of 1 major coronary artery (e.g. >2 months) and ≥50% narrowing of the luminal diameter by coronary angiography or CT angiography of at least 1 additional major coronary artery,
c) high-risk single-vessel coronary artery disease, such as e.g. defined as the presence of ≥50% narrowing of the luminal diameter of one major coronary artery (e.g. by coronary angiography or CT angiography in patients not revascularised) and at least one of the following:
a positive non invasive stress test, such as e.g. confirmed by either:
a positive ECG exercise tolerance test in patients without left bundle branch block, Wolff-Parkinson-White syndrome, left ventricular hypertrophy with repolarization abnormality, or paced ventricular rhythm, atrial fibrillation in case of abnormal ST-T segments,
a positive stress echocardiogram showing induced regional systolic wall motion abnormalities,
a positive nuclear myocardial perfusion imaging stress test showing stress induced reversible perfusion abnormality,
patient discharged from hospital with a documented diagnosis of unstable angina pectoris (e.g. ≥2-12 months),
d) previous ischemic or haemorrhagic stroke (e.g. >3 months),
e) presence of carotid artery disease (e.g. symptomatic or not), such as e.g. documented by either:
imaging techniques with at least one lesion estimated to be ≥50% narrowing of the luminal diameter,
prior percutaneous or surgical carotid revascularization,
f) presence of peripheral artery disease, such as e.g. documented by either:
previous limb angioplasty, stenting or bypass surgery,
previous limb or foot amputation due to macrocirculatory insufficiency,
angiographic evidence of peripheral artery stenosis ≥50% narrowing of the luminal diameter in at least one limb (e.g. definition of peripheral artery: common iliac artery, internal iliac artery, external iliac artery, femoral artery, popliteal artery),

### Condition II:

impaired renal function (e.g. with or without CV co-morbidities), such as e.g. defined by:
impaired renal function (e.g. as defined by MDRD formula) with an estimated glomerular filtration rate (eGFR) 15-45 mL/min/1.73 m² with any urine albumin creatinine ratio (UACR), or.
impaired renal function (e.g. as defined by MDRD formula) with an with an estimated glomerular filtration rate (eGFR) ≥45-75 mL/min/1.73 m² with an urine albumin creatinine ratio (UACR) >200 mg/g creatinine or >200 mg/l (milligram albumin per liter of urine) or >200μg/min (microgram albumin per minute) or >200 mg/24 h (milligram albumin per 24 hours);
are treated over a lengthy period (e.g. for at least 12-48 months, preferably at least about 20-24months) with linagliptin (preferably 5 mg per day, administered orally, preferably in form of a tablet, optionally in combination with one or more other active substances, e.g. such as those described herein) and compared with patients who have been treated with placebo (as add-on therapy on top of standard of care).

Evidence of the therapeutic success compared with patients who have been treated with placebo can be found in non-inferiority or superiority compared to placebo, e.g. in the (longer) time taken to first occurrence of cardio-or cerebrovascular events, e.g. time to first occurrence of any of the following components of the composite CV endpoint: cardiovascular death (including fatal stroke, fatal myocardial infarction and sudden death), non-fatal myocardial infarction (excluding silent myocardial infarction), non-fatal stroke, and (optional) hospitalisation e.g. for heart failure; and/or
in the (longer) time taken to first occurrence of renal microvascular events, e.g. time to first occurrence of any of the following components of the composite renal endpoint: renal death, sustained end-stage renal disease (ESRD), and sustained decrease of 40% or more (or 50% or more) in eGFR.

Further therapeutic success can be found in the (smaller) number of or in the (longer) time taken to first occurrence of any of: cardiovascular death, (non)-fatal myocardial infarction, silent MI, (non)-fatal stroke, hospitalisation for unstable angina pectoris, hospitalisation for coronary revascularization, hospitalisation for peripheral revascularization, hospitalisation for (congestive) heart failure, all cause mortality, renal death, sustained end-stage renal disease, loss in eGFR, new incidence of macroalbuminuria, progression in albuminuria, progression in CKD, need for anti-retinopathy therapy; or improvement in albuminuria, renal function, CKD; or improvement in cognitive function or prevention of/protection against accelerated cognitive decline.

Cognitive functions can be assessed by standardized tests as measure of cognitive functioning such as e.g. by using the Mini-Mental State Examination (MMSE), the Trail Making Test (TMT) and/or the Verbal Fluency Test (VFT).

Additional therapeutic success (compared to placebo) can be found in greater change from baseline in HbA1c and/or FPG. Further additional therapeutic success can be found in greater proportion of patients on study treatment at study end maintain glycemic control (e.g. HbA1c </=7%).

Further additional therapeutic success can be found in greater proportion of patients on study treatment who at study end maintain glycemic control without need for additional antidiabetic medication (during treatment) to obtain HbA1c </=7%.

Further additional therapeutic success can be found in lower proportion of patients on study treatment initiated on insulin or treated with insulin or in lower dose of insulin dose used.

Further additional therapeutic success can be found in lower change from baseline in body weight or greater proportion of patients with ≤2% weight gain or lower proportion of patients with ≥2% weight gain at study end.

Respective subgroup analysis may be made in this study for patients having chronic kidney disease (CKD) such as e.g. up to stage 3 and/or having estimated glomerular filtration rate (eGFR; mL/minute/1.73 m²) levels down to 45, or down to 30, such as for patients with (chronic) renal impairment of moderate stage (CKD stage 3, eGFR 30-60), particularly of mild-to-moderate stage (CKD stage 3a) such as having eGFR levels 45-59 or of moderate-to-severe stage (CKD stage 3b) such as having eGFR levels 30-44; optionally with or without micro-or macroalbuminuria.

Over two thirds (71%) of the total participants of above study are categorized as having a (renal) prognosis of high risk (27.2%) or very high risk (43.5%) by eGFR and albuminuria categories at baseline:

Prognosis of CKD in study population by eGFR and albuminuria categories

### High Risk:

UACR (mg/g) >300 and eGFR (ml/min/1.73 m²) >60, or
UACR (mg/g) 30-299 and eGFR (ml/min/1.73 m²) 45-59, or
UACR (mg/g) <30 and eGFR (ml/min/1.73 m²) 30-44;

### Very High Risk:

UACR (mg/g) >300 and eGFR (ml/min/1.73 m²) 45-59 or 30-44 or <30, or
UACR (mg/g) 30-299 and eGFR (ml/min/1.73 m²) 30-44 or <30, or
UACR (mg/g) <30 and eGFR (ml/min/1.73 m²) <30.

Respective subgroup analysis may be also made in this study for patients having renal prognosis of high risk or very high risk as defined above.

### Results

### Summary Conclusions

Trial Patients and Compliance with Trial Protocol:

The population of the trial was as intended, allowing the assessment of cardiovascular and renal outcomes in a population frequently encountered in clinical practice. The 6979 treated patients represented major geographical regions and races. As per the inclusion criteria, the patients all had a high risk of CV events.

The majority of patients (74%) had prevalent kidney disease at baseline, defined as eGFR <60 mL/min/1.73m2 or urine albumin creatinine ratio (UACR) ≥300 mg/g. More than half (57%) of the patients had both established macrovascular disease and albuminuria. Overall, 71% of the patient population were considered to be at high risk or very high risk for adverse kidney events on the basis of their eGFR and albuminuria status (KDIGO risk categories).

Overall, all demographic and clinical characteristics were balanced between the treatment groups. Less than 1% of patients were lost to follow-up for vital status. Premature discontinuation of trial medication was slightly higher in the placebo group than in the linagliptin group. Very few important protocol violations were reported in either treatment group, and >99% of patients were included in the per protocol analysis set. The median time in trial was 2.2 years in both the linagliptin and placebo groups. The median treatment exposure was 1.9 and 1.8 years in the linagliptin and placebo groups, respectively.

### Efficacy:

### Primary and Key Secondary Endpoints

A total of 854 patients were reported with an adjudication-confirmed primary endpoint event (first occurrence of any of the following adjudication-confirmed components: CV death, non-fatal MI, or non-fatal stroke [3P-MACE]). There were 434 patients (12.4%) with an event in the linagliptin group and 420 patients (12.1%) in the placebo group. The hazard ratio (HR) based on Cox proportional hazards regression model for linagliptin vs. placebo was 1.02 (95% CI 0.89, 1.17).

Linagliptin was therefore demonstrated to be non-inferior to placebo with an upper bound of the 95% CI of below 1.3 and not superior to placebo.

A total of 633 patients were reported with an adjudication-confirmed key secondary endpoint event (first occurrence of any of the following adjudication-confirmed components: renal death, sustained ESRD (End Stage Renal Disease) or sustained decrease of 40% or more in eGFR (estimated Glomerular Filtration Rate) from baseline [composite renal endpoint 1]). There were 327 patients (9.4%) with an event in the linagliptin group and 306 patients (8.8%) in the placebo group. The HR based on Cox regression for linagliptin vs. placebo was 1.04 (96% CI 0.88, 1.23). Linagliptin was therefore found to be not superior to placebo. Despite the positive trend observed for the analyses of combined sustained ESRD or renal death (linagliptin: 136 patients [3.9%], placebo: 154 patients [4.4%] with an event), the HR of 0.87 was not statistically significant.

Sensitivity analyses of the primary and key secondary endpoints were performed on the PPS (Per Protocol Set), the OS (On-treatment Set), the TS (Treated Set) +30 days censoring approach and the TS (Treated Set) +0 days censoring approach and all results were consistent with the findings of the main analyses.

The primary endpoint was also analysed across a range of subgroups and in general consistent results for the treatment effect were observed across the subgroups. No significant difference in the treatment effect was observed between patients with or without insulin treatment at baseline or in other subgroups of interest such as patients with or without prevalent kidney disease or across eGFR categories at baseline.

Cox regression for time to first 3P-MACE and composite
renal endpoint 1 events, linagliptin vs. placebo-TS

| | Lingagliptin | Placebo |
|---|---|---|
| Total patients in TS, N (100%) | 3494 | 3485 |
| 3-point MACE, N (%) [incidence rate/1000 y] | 434 (12.4) [57.7] | 420 (12.1) [56.3] |
| HR vs. placebo 95% CI; alpha-level = 2.5% | 1.02 (0.89. 1.17) | |
| 99% CI; alpha-level = 0.5% | (0.86, 1.22) | |
| p-value for HR ≥ 1.3 (1-sided) | 0.0002 | |
| p-value for HR ≥ 1.0 (1-sided) | 0.6301 | |
| Composite renal endpoint 1, N (100%) | 327 (9.4) [48.9] | 306 (8.8) [46.6] |
| [incidence rate/1000 y] | | |
| HR vs. placebo (95% CI; alpha-level = 2.5%) | 1.04 (0.89, 1.22) | |
| (96% CI; alpha-level = 2.0%) | (0.88, 1.23) | |
| p-value for HR ≥ 1.0 (1-sided) | 0.6918 | |

### Tertiary Endpoints

### CV Outcomes

For the endpoints of 4P-MACE, CV death, all-cause mortality, and MI-related endpoints, no significant differences were observed between the linagliptin and placebo groups.

### Cerebrovascular Results

For the endpoints of fatal and non-fatal stroke and transient ischaemic attack, no significant differences were observed between the linagliptin and placebo groups.

### Heart Failure Endpoint Results

For the endpoints of hospitalisation for heart failure; CV death or hospitalisation for heart failure;

all-cause mortality or hospitalisation for heart failure; and heart failure AEs, no significant differences were observed between the patients treated with linagliptin and those on placebo. There was also no difference observed in the subgroups of patients with or without a history of heart failure nor those with or without prevalent kidney disease. For patients with or without insulin use at baseline, a significant subgroup by treatment interaction favouring linagliptin in patients without insulin vs a neutral result in those on insulin was observed.

| | Linagliptin | Placebo |
|---|---|---|
| Total patients in TS, N (100%) | 3494 | 3485 |
| Hospitalisation for heart failure, N (%) | 209 (6.0) [27.7] | 226 (6.5) [30.4] |
| [incidence rate/1000 y] | | |
| HR vs. placebo (95% CI; alpha-level = 2.5%) | 0.90 (0.74, 1.08) | |
| CV death, N (%) [incidence rate/1000 y] | 255 (7.3) [32.6] | 264 (7.6 [34.0] |
| HR vs. placebo (95% CI; alpha-level = 2.5%) | 0.96 (0.81, 1.14) | |
| All-cause mortality, N (%) [incidence rate/1000 y] | 367 (10.5) [46.9] | 373 (10.7) [48.0] |
| HR vs. placebo (95% CI; alpha-level = 2.5%) | 0.98 (0.84, 1.13) | |

### Kidney Outcome Results

There was no significant difference between the treatment groups for sustained ESRD or renal death.

No significant difference was observed between the linagliptin group and the placebo group for the adjudicated composite renal endpoint 2 (renal death, sustained ESRD, or sustained decrease of 50% or more in eGFR from baseline) or for composite renal endpoint 3 (renal death, sustained ESRD, or sustained decrease of 30% or more in eGFR (MDRD) from baseline accompanied by eGFR (MDRD) <60 ml/min/m2), the latter was not adjudicated, but only based on central laboratory data.

There was also no significant difference between the treatment groups for the other combined endpoints of sustained decrease of 30% or more in eGFR (MDRD) from baseline accompanied by eGFR (MDRD) <60 ml/min/m2; and renal death, sustained ESRD or CV death.

### Microvascular Results

For the endpoint of time to composite microvascular outcome 1 (renal death, sustained ESRD, sustained 50% decrease or more in eGFR from baseline, albuminuria progression, use of retinal photocoagulation or intravitreal injection(s) of an anti-VEGF therapy for diabetic retinopathy or vitreous haemorrhage or diabetes-related blindness), the risk was significantly reduced in the linagliptin group compared with the placebo group. The difference was driven mainly by a lower incidence of albuminuria progression in the linagliptin group. For the endpoints of composite microvascular outcome 2 and 3, using an eGFR decrease of 40% and 30%, respectively, the risk was also significantly reduced in the linagliptin group compared with the placebo group. These differences were also driven by the lower incidence of albuminuria progression in the linagliptin group.

### Albuminuria Related Results

For the endpoint of albuminuria progression, the risk was significantly reduced in the linagliptin group compared with the placebo group. New incidence of micro-and macroalbuminuria were both directionally congruent with albuminuria-progression, with a larger reduction observed in patients with prevalent albuminuria or prevalent kidney disease. A statistically significant and clinically meaningful reduction in UACR was observed in the linagliptin group up to Week 132, compared with placebo, and a greater magnitude of effect was seen in patients with prevalent kidney disease at baseline. Further endpoints of eGFR changes over time and transition in chronic kidney disease (CKD) status were evaluated, and no clinically meaningful differences were observed between the treatment groups.

### Further Tertiary Endpoint Results

The endpoints of stent thrombosis, hospitalisation for peripheral vascularisation, and retinopathy-related endpoints all showed no significant difference between the linagliptin and placebo treatment groups.

### Other Endpoints

The difference between the treatment groups in HbA1c changes from baseline over time was significant up to the Week 132 visit. The proportion of patients who achieved glycaemic control at the study end visit, without additional antidiabetic medication or an increase in background antidiabetic medication was significantly greater in the linagliptin group than in the placebo group. A similar pattern was observed for the proportion of patients who achieved glycaemic control at the end of the study irrespective of antidiabetic medication (linagliptin: 1012 patients [29.0%, placebo: 685 patients [19.7%]). A similar pattern was observed for fasting plasma glucose (FPG), with significant differences observed between the treatment groups up to Week 84.

No clinically meaningful differences were observed between the treatment groups for changes in body weight or waist circumference over the course of the study. In patients without insulin use at baseline, time to onset of intensification or initiation of insulin was later in patients treated with linagliptin than in patients treated with placebo.

### Conclusions:

This trial evaluated the effect of linagliptin on cardiovascular and kidney outcomes in patients with type 2 diabetes who were at high cardiovascular risk. Unlike other completed CV outcome trials with DPP-4 inhibitors, this trial included a particularly high proportion of patients with prevalent kidney disease in addition to those with established macrovascular disease, thereby investigating a highly vulnerable population for cardiovascular and renal events. In this trial, linagliptin was shown to be non-inferior to placebo on top of standard of care for time to first occurrence of CV death, non-fatal MI, or non-fatal stroke (3P-MACE). There was also no increased risk for hospitalisation for heart failure or any other heart failure endpoint. Linagliptin was comparable to placebo in time to first occurrence of renal death, sustained ESRD or sustained decrease of 40% or more in eGFR from baseline. Linagliptin reduced albuminuria as well as HbA1c, without increasing the risk for hypoglycaemia. Linagliptin was well tolerated overall and the safety profile in this study was consistent with the known profile of the drug. In summary, cardiovascular and renal safety of linagliptin have been demonstrated in a CV high risk population with established macrovascular and/or prevalent kidney disease.

In further more detail:

Effects of Linagliptin on Heart Failure Outcomes in Patients with Type 2 Diabetes and Cardio-Renal Disease in the present Cardiovascular and Renal Outcomes Trial

Background and aims: People with type 2 diabetes (T2D) are at increased risk for hospitalization for heart failure (hHF), particularly in the setting of concomitant cardiovascular (CV) and/or kidney disease. Some, but not all, dipeptidyl peptidase-4 (DPP-4) inhibitors have been associated with increased hHF in high-CV risk populations. Here we present analyses of HF outcomes with the DPP-4 inhibitor linagliptin (LINA) vs. placebo (PBO) from a Cardiovascular and Renal Outcomes Trial assessing cardiovascular safety and renal microvascular outcome in patients with type 2 diabetes at high vascular risk, a large CV outcomes trial that enrolled participants with T2D at high risk for hHF due to concomitant CV and/or chronic kidney disease. Materials and methods: People with T2D and concomitant CV and/or kidney disease were randomized to receive LINA 5 mg, or PBO once daily (1:1), on top of standard of care. All hHF, CV outcomes, and deaths were centrally adjudicated, with individual and composite HF-related outcomes analyses comparing LINA vs. placebo. Investigator-reported HF-related adverse events, whether or not confirmed by adjudication, were also analyzed. A Cox proportional hazards model adjusting for region and history of HF was used for analyses of first events. Recurrent hHF events were analyzed using a negative binomial model. The effect of LINA on hHF was compared across baseline subgroups including history of HF, insulin use, age < or ≥65 years, eGFR < or ≥60 ml/min/1.73m², and geographical region.

Results: This Cardiovascular and Renal Outcomes Trial enrolled 6979 participants with mean age 65.9 yrs, BMI 31.3 kg/m², eGFR 54.6 ml/min/m² and HbA1c 8.0%; 62.9% men; 58.5% had ischemic heart disease and 26.8% a history of HF. Median follow up was 2.2 yrs with trial completeness and vital status availability of 98.6% and 99.7%, respectively. LINA did not affect the risk of time to first event of hHF (LINA 209/3494, 27.7/1000 pt-yrs vs PBO 226/3485, 30.4/1000pt-yrs; HR 0.90 [95% CI 0.74, 1.08]). Consistently neutral effects of LINA vs PBO were observed across a series of individual and composite HF-related outcomes, recurrent hHF events, and initiation of diuretic therapy (FIG. 2, Effects of LINA vs PBO on individual and composite HF-related outcomes, recurrent hHF events, initiation of diuretic therapy and in subgroups of interest.). Across subgroups of interest, heterogeneity was observed by baseline insulin use, where LINA resulted in a nominally significant reduction in hHF among those without but not with baseline insulin use (p_{interaction}=0.036), and by region with nominally significant reductions in hHF with LINA in North America and Asia (p_{interaction}=0.037).

Conclusion: In a large, international CV outcome trial in patients with T2D and concomitant CV and/or kidney disease, linagliptin did not increase the risk for hHF or other HF-related outcomes, including among participants with and without a history of HF.

Also, in further more detail:

Effect of Linagliptin on Kidney and Cardiovascular Outcomes in Patients with Type 2 Diabetes and Kidney Disease in the present Cardiovascular and Renal Outcomes Trial

Background: Type 2 diabetes (T2D) is a common cause of end stage kidney disease (ESKD) so the effects of glucose-lowering therapies on kidney outcomes are of great interest, especially in people with CKD.

Methods: The present Cardiovascular and Renal Outcomes Trial randomized people with T2D and i) concomitant CV disease with UACR >30 mg/g or ii) prevalent CKD (i.e. GFR <45 ml/min/1.73m2 and/or UACR >200 mg/g) to receive the DPP-4 inhibitor linagliptin (5 mg) or placebo once daily in a double-blind fashion. The primary CV endpoint was 3P-MACE, with a key secondary kidney endpoint (adjudicated ESKD, renal death, or sustained ≥40% decrease in eGFR from baseline) and other renal outcomes (including albuminuria and eGFR slope) also assessed. Subgroups were assessed by baseline kidney function (eGFR ≥/<45 ml/min/1.73m2and eGFR ≥/<30, 45 or 60 ml/min/1.73m2).

Results: 6979 participants (mean age 65.9 yrs, HbA1c 8.0%, eGFR 54.6 ml/min/1.73m2, 43% eGFR ≤45, and 80.3% UACR >30 mg/g) from 660 centers across 27 countries were followed-up for median 2.2 yrs. Linagliptin reduced albuminuria progression and albuminuria levels; eGFR-slope (Table 4) was unaffected. Rates of the secondary kidney endpoint (HR 1.04 [0.89, 1.22]), renal death, or sustained ESKD (0.87 [0.69, 1.10]), and renal death, sustained ESKD, or sustained ≥doubling of se-creatinine (0.92 [0.77, 1.11]), as well as 3P-MACE and hospitalization for heart failure (Table 4) were also similar between randomized groups. All outcomes occurred at higher incidence rates in those with reduced eGFR, however, results were consistent across kidney function subgroups (all p heterogeneity >0.1).

Conclusions: Linagliptin slowed progression of albuminuria, without affecting long-term eGFR slope or other kidney outcomes. Linagliptin also demonstrated CV safety including in patients with advanced CKD where clinical evidence has been particularly scarce.

TABLE 4

| Effects on kidney | | | | | | |
|---|---|---|---|---|---|---|
| surrogate | | | | | | |
| parameters | Linagliptin | | Placebo | | | |
| Albuminuria | n (%) | Rate/100 | n (%) | Rate/100 | HR for progression | P-value |
| progression¹ | | patient- | | patient- | 0.86 (0.78, 0.95) | 0.0034 |
| (n = 4291) | | years | | years | | |
| | 763 | 21.36 | 819 | 24.54 | | |
| | (35.3) | | (38.5) | | | |

| Absolute change in | Baseline, median (IQR) | | Difference | Difference | p-value |
|---|---|---|---|---|---|
| UACR², mg/g | 158.41 | 154.87 | at week 36 | at week 84 | |
| | (43.36, 684.07) | (42.48, 706.19) | 0.87 | 0.88 | Both p < |
| | (n = 3258) | (n = 3231) | (0.81, | (0.82, | 0.01 |
| | | | 0.93) | (0.95) | |

| eGFR slope (MDRD), | eGFR-slope from baseline to last value | | | | Between-group | |
|---|---|---|---|---|---|---|
| estimate ± SE (n = 6740) | on treatment/year | | | | difference | |
| | −2.459 ± 0.106 | | −2.284 ± 0.108 | | −0.175 ± 0.151 | 0.2485 |
| Effects on CV and | Linagliptin | | Placebo | | HR | P-value |
| kidney outcomes | (N = 3494) | | (N = 3485) | | (95% Cl) | |
| | n (%) | Rate/100 | n (%) | Rate/100 | | |
| | | patient- | | patient- | | |
| | | years | | years | | |
| 3P-MACE (CV death, | 434 | 4.69 | 420 | 5.63 | 1.02 (0.89, 1.17) | 0.7398 |
| non-fatal myocardial | (12.4) | | (12.1) | | | |
| infarction, or non- | | | | | | |
| fatal stroke) | | | | | | |
| eGFR < 45 (n = 3000) | 250 | 7.61 | 241 | 7.49 | 1.02 (0.85, 1.21) | 0.9361 |
| | (16.6) | | (16.2) | | | (p-for |
| eGFR ≥ 45 (n = 3979) | 184 | 4.34 | 179 | 4.23 | 1.03 (0.84, 1.26) | interaction) |
| | (9.3) | | (9.0) | | | |
| Hospitalized heart | 209 | 2.77 | 226 | 3.04 | 0.90 (0.74, 1.08) | 0.2635 |
| failure | (6.0) | | (6.5) | | | |
| eGFR < 45 (n = 3000) | 135 | 4.13 | 153 | 4.81 | 0.87 (0.69, 1.10) | 0.5933 |
| | (8.9) | | (10.3) | | | (p-for |
| | | | | | | interaction) |
| eGFR ≥ 45 (n = 3979) | 74 | 1.73 | 73 | 1.72 | 0.97 (0.70, 1.34) | |
| | (3.7) | | (3.7) | | | |
| Key secondary | 327 | 4.89 | 306 | 4.66 | 1.04 (0.89, 1.22) | 0.6164 |
| kidney endpoint | (9.4) | | (8.8) | | | |
| (Renal death, | | | | | | |
| sustained ESKD or | | | | | | |
| sustained decrease | | | | | | |
| of 40% or more in | | | | | | |
| eGFR from baseline) | | | | | | |
| eGFR < 45 (n = 3000) | 222 | 7.83 | 219 | 7.93 | 0.97 (0.80, 1.17) | 0.2398 |
| | (14.7) | | (14.7) | | | (p-for |
| eGFR ≥ 45 (n = 3979) | 105 | 2.72 | 87 | 2.29 | 1.19 (0.89, 1.58) | interaction) |
| | (5.3) | | (4.4) | | | |
| Renal death, or | 136 | 1.78 | 154 | 2.04 | 0.87 (0.69, 1.10) | 0.2371 |
| sustained ESKD | (3.9) | | (4.4) | | | |
| eGFR < 45 (n = 3000) | 124 | 3.78 | 146 | 4.54 | 0.82 (0.64, 1.04) | 0.2004 |
| | (8.2) | | (9.8) | | | (p-for |
| eGFR ≥ 45 (n = 3979) | 12 | 0.28 | 8 | 0.19 | 1.50 (0.61, 3.67) | interaction) |
| | (0.6) | | (0.4) | | | |
| Renal death, | 219 | 3.21 | 229 | 3.43 | 0.92 (0.77, 1.11) | 0.4011 |
| sustained ESKD, or | (6.3) | | (6.6) | | | |
| sustained ≥ doubling | | | | | | |
| of se-creatinine from | | | | | | |
| baseline³ | | | | | | |
| eGFR < 45 (n = 3000) | 165 | 5.69 | 180 | 6.38 | 0.87 (0.70, 1.07) | 0.3261 |
| | (10.9) | | (12.1) | | | (p-for |
| eGFR ≥ 45 (n = 3979) | 54 | 1.38 | 49 | 1.27 | 1.08 (0.73, 1.59) | interaction) |
| | (2.7) | | (2.5) | | | |

HR based on Cox regression analyses in patients treated with ≥1 dose of study drug.
¹change from normoalbuminuria to micro-/macroalbuminuria, or change from microalbuminuria to macroalbuminuria.
²gMean ratio of relative change for linagliptin versus placebo.
³doubling of se-creatinine accompanied by eGFR < 60 ml/min/1.73m2 (MDRD).

Also, in further more detail:

### Study Design

The design of the present Cardiovascular and Renal Outcomes Trial has previously been described (Rosenstock J, Perkovic V, Alexander J et al. Rationale, Cardiovasc Diabetol. 2018;17:39, the disclosure of which is incorporated herein). In brief, this was a randomized, double-blind, placebo-controlled clinical trial conducted in 660 centers across 27 countries, and aimed to continue until at least 611 participants had an adjudication-confirmed primary outcome event.

### Study Participants

Adults with type 2 diabetes, HbA1c 6.5-10.0% inclusive, and high CV risk were eligible for inclusion. High risk was defined as i) high levels of albuminuria (micro-or macro-albuminuria, defined as urinary albumin: creatinine ratio (UACR) >30 mg/g or equivalent) AND established macrovascular disease, and/or ii) impaired renal function (eGFR 45-75 ml/min/1.73m² and UACR >200 mg/g or equivalent, OR eGFR 15-45 regardless of UACR). Macrovascular disease eligibility criteria was based on documented and confirmed history of myocardial infarction, coronary artery disease, stroke, carotid artery disease, or peripheral artery disease. Participants with end-stage kidney disease (ESKD), defined as eGFR <15 or requiring maintenance dialysis, were excluded.

### Study Procedures

Eligible individuals were randomized 1:1 to once-daily double-blind oral linagliptin 5 mg or matching placebo. Treatment assignment was determined by computer-generated random sequence with stratification by geographical region (North America, Latin America, Europe [plus South Africa], and Asia). Following randomization, participants returned for study visits after 12 weeks and then every 24 weeks until study-end. A final follow-up visit was scheduled 30 days after the end of treatment. In an attempt to maintain glycemic equipoise, investigators were encouraged to monitor and use additional medication for glycemic control (except DPP-4inhibitors, GLP1 receptor agonists, and SGLT2 inhibitors) according to applicable standard of care throughout the trial, independent of study treatment assignment that remained blinded. Treatment of other CV risk factors was encouraged in accordance with applicable guidelines and current standards of care. Patients who prematurely discontinued study medication were followed for ascertainment of CV and key secondary kidney outcome events, and attempts were made to collect vital status information on every randomized patient at study completion, in compliance with local law and regulations.

### Study Outcomes

The primary outcome was defined as the time to first occurrence of CV death, non-fatal myocardial infarction (MI) or non-fatal stroke (3-point major adverse CV event; MACE). The key secondary outcome was defined as time to first occurrence of a composite of adjudication-confirmed renal death, ESKD, or a sustained decrease of >40% in eGFR from baseline. Further outcomes include time to hospitalization for HF, all-cause death, the composite of renal death or ESKD, and a microvascular composite outcome that included albuminuria, hard kidney outcomes and major ocular events. Additional outcomes were progression in albuminuria category and change from baseline in HbA1c. Safety was assessed based on adverse events reported.

### Results

Study participants 6991 patients were randomized of whom 6979 received at least one dose of study drug and are included in the primary analysis. Overall, 98.7% of participants completed the study, with 25.6% of patients prematurely discontinuing study drug. Vital status was available for 99.7% of patients at study completion. Baseline clinical characteristics were balanced between groups and patients were well managed overall with regard to CV and kidney disease risk factors (Table 5): 57% had established CV disease, 74% prevalent kidney disease (defined as eGFR <60ml/min/1.73m² and/or UACR >300 mg/g creatinine) and 33% both CV and kidney disease. 15.2% had eGFR <30 ml/min/1.73m². Median treatment duration and observation time were 1.9 and 2.2 years, respectively.

### Glycemic Control

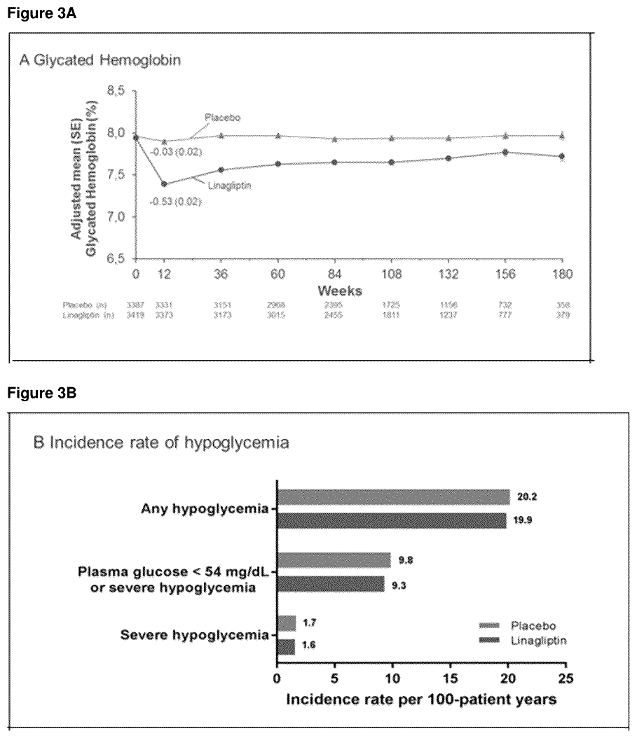

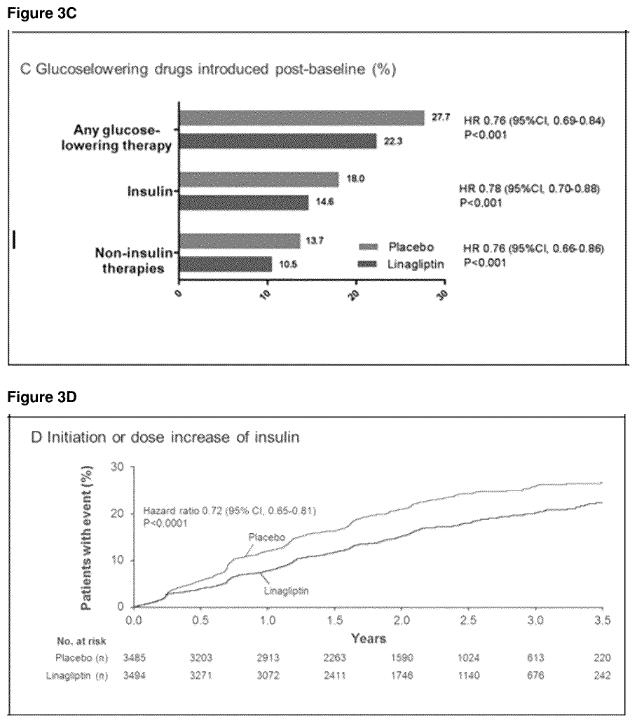

After 12 weeks of treatment, the adjusted mean difference in glycated hemoglobin with linagliptin versus placebo was-0.51% (95% CI-0.55 to-0.46) (FIG. 3A), with an overall difference over the full study duration of-0.36% (95% CI-0.42,-0.29; based on least square means), without increase in overall hypoglycemia risk (FIG. 3B) and despite a higher use of additional glucose-lowering medications (FIG. 3C) in the placebo group which had more patients initiating or increasing doses of pre-existing insulin therapy (FIG. 3D).

### Weight, BP, LDL-C

Overall, changes in weight, systolic and diastolic blood pressure and low-/high density lipoprotein cholesterol were no different between groups. New introductions of blood-pressure lowering medications, anticoagulants or LDL-cholesterol lowering drugs were similar between the linagliptin and placebo arms.

### Cardiovascular Outcomes and Mortality

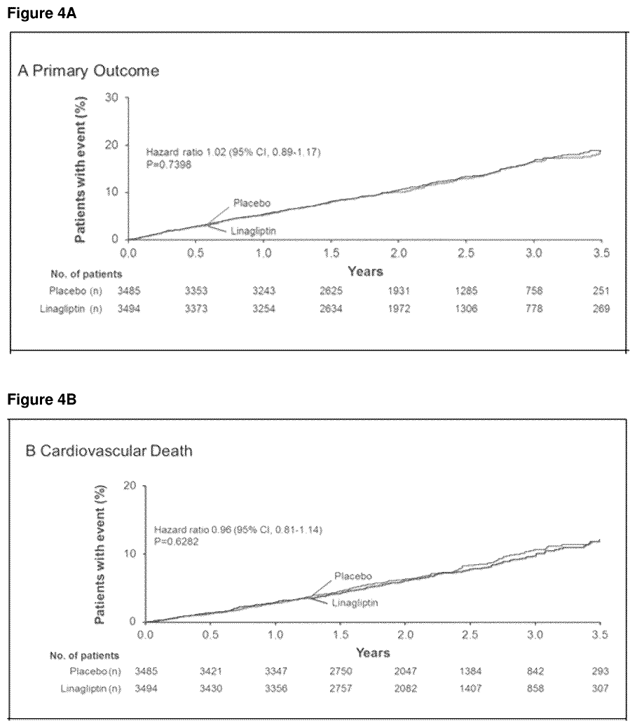

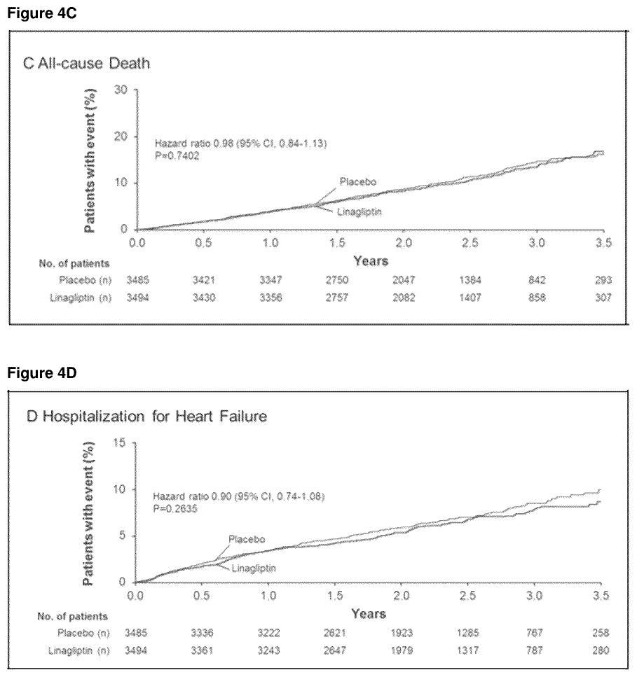

The primary composite 3-point MACE occurred in 434/3494 (12.4%) patients randomized to linagliptin (5.77 per 100 person-years) and 420/3485 (12.1%) patients randomized to placebo (5.63 per 100 person-years). Linagliptin was noninferior to placebo (HR1.02 [95% CI 0.89, 1.17], p_{noninferiority}=0.0002; Table 6 and FIG. 4A), but did not achieve superiority (p=0.7398). Pre-specified sensitivity analyses of the primary outcome yielded consistent results. Overall, the risk for the primary outcome was consistent across pre-specified subgroups (Table 7), apart from some indication of heterogeneity for subgroups of glycated haemoglobin and use of calcium channel blockers. Four-point MACE occurred in 463/3493 (13.3%) vs 459/3485 (13.2%), in the linagliptin and placebo arm respectively (HR 1.00 [95% CI 0.88, 1.13], p=0.9598).Similarly, no significant differences were observed for the risk of individual component outcomes, including CV death (Table 6; FIG. 4B). Death from any cause occurred in similar proportions among linagliptin (10.5%, 4.69 per 100 person-years) and placebo treated participants (10.7%, 4.80 per 100 patient-years) (HR 0.98 [95% CI 0.84, 1.13], p=0.7402) (Table 6; FIG. 4C).

### Kidney and Microvascular Outcomes

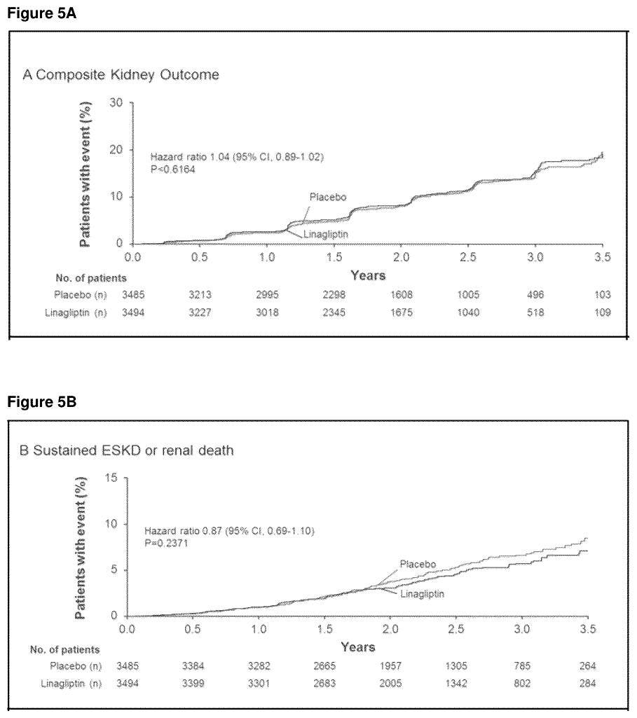

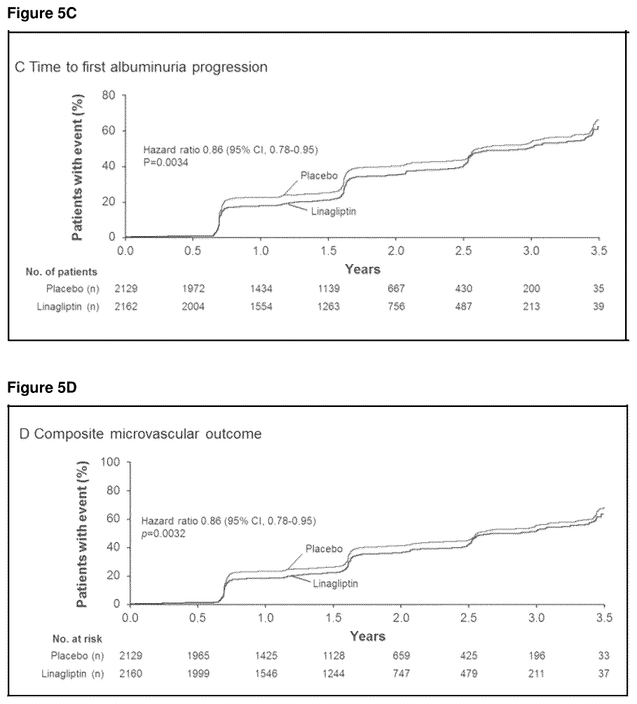

The key secondary kidney outcome occurred in similar proportions among linagliptin (9.4%, 4.89per 100 person-years) and placebo treated participants (8.8%, 4.66 per 100 patient-years) arms (HR 1.04 [95% CI 0.89, 1.22], p=0.62) (Table 6, FIG. 5A); pre-specified sensitivity and subgroup analyses demonstrated similar results, apart from some indication of heterogeneity for duration of type 2 diabetes; Table 8. The composite of renal death, sustained ESKD, or sustained decrease of 50% or more in eGFR showed similar results (Table 6). An additional outcome of ‘hard kidney events’ comprising a composite of sustained ESKD or death due to kidney disease was also not statistically different (3.9%, 1.78 per 100 patient-years vs 4.4%, 2.04 per 100 patient-years; HR 0.87 [95% CI 0.69, 1.10], p=0.24 [Table 6, FIG. 5B]).

Progression of albuminuria category (i.e. change from normal albuminuria to microalbuminuria, or change from microalbuminuria to macroalbuminuria) occurred less frequently in the linagliptin (763/2162 [35.3%], 21.4 per 100 patient-years) than in the placebo arm (819/2129 [38.5%], 24.5 per 100 patient-years); HR 0.86 (95% CI 0.78, 0.95), p=0.0034. (Table 6, FIG. 5C). Another pre-specified microvascular composite outcome including both kidney and major ocular events (renal death, ESKD, or sustained 50% reduction in eGFR, albuminuria progression, retinal laser coagulation or anti-VEGF injection for diabetic retinopathy, vitreous haemorrhage, or diabetes-related blindness), occurred less frequently in linagliptin treated participants than those allocated to placebo (HR 0.86 [95% CI 0.78, 0.95], p=0.0032) (Table 6, FIG. 5D). Ocular outcomes were not statistically different between the linagliptin and placebo arms (HR 0.73 [95% CI 0.47, 1.12], p=0.1472), Table 9, Table 10.

### Heart Failure

Hospitalization for HF occurred in 209/3494 patients randomized to linagliptin (6.0%; 2.77 per 100 person-years) and 226/3485 patients randomized to placebo (6.5%; 3.04 per 100 person-years), with no significant difference between the two treatment groups (HR 0.90 [95% CI 0.74, 1.08], p=0.2635) (Table 6; FIG. 4D). Pre-specified sensitivity analyses yielded consistent results. The composite outcome of time to first event of CV death or hospitalization for HF, occurred in 406/3494 patients randomized to linagliptin (11.6%; 5.37 per 100 person-years) and 422/3485 patients randomized to placebo (12.1%; 5.66 per 100 person-years), also with no significant difference between the two treatment groups (HR 0.94 [95% CI 0.82,1.08], p=0.3881).

In addition to that there was no difference for linagliptin versus placebo for the composite outcomes of hHF or death (406 vs. 422 events; HR 0.94, 95% CI 0.82, 1.08), there was also no difference for linagliptin versus placebo for hHF or all-cause mortality (499 vs. 518 events; HR 0.95, 95% CI 0.84, 1.07, investigator reported HF events (243 vs 271 events; HR 0.87 [0.73, 1.03]), or the combination of time to first event of investigator reported events or adjudicated hHF (305 vs 326 events, HR 0.92 [0.79, 1.08]). In recurrent events analysis, the cumulative number of hHF events (first+recurrent) was not different between linagliptin and placebo groups (326 vs. 359 events; rate ratio 0.94, 95% CI 0.75, 1.25) and in total 60 (1.7%) participants in the linagliptin group and 78 (2.2%) in the placebo group had ≥2 hHF events. New introduction of loop diuretics was not different between linagliptin and placebo (318/2530 vs 324/2461 participants, HR 0.94, 95% CI 0.81, 1.10), with no difference in the composite outcome of new initiation of loop diuretics or hHF (330/2530 vs 333/2461 participants, HR 0.95, 95% CI 0.82, 1.11). Pre-specified and post-hoc defined sensitivity analyses of hHF yielded consistent results with the primary analysis.

The incidence of hHF varied substantially across subgroups defined by baseline characteristics (Table 11). However, among the subset of participants with or without a history of HF at baseline, there were no significant differences observed between the treatment groups in hHF (p-for interaction 0.8104). Also, no heterogeneity was observed for the effects of the randomized treatment assignment by baseline HF history for CV death (p_{interaction} 0.763), or the primary outcome 3-point MACE (p_{interaction} 0.9588).

There was statistical heterogeneity of linagliptin effects on hHF by some subgroups analyzed (Table 11): by region (Table 11); by insulin use at baseline (Table 11); and by baseline BP. Statistically significant lower risk of hHF with linagliptin than placebo was observed for those enrolled from North America or Asia (p_{interaction}=0.0368), and those not treated with insulin at baseline (p_{interaction}=0.0360). In addition, heterogeneity of hHF effect of linagliptin was also observed by baseline systolic BP (SBP), with statistically lower risk of hHF with linagliptin than placebo in the subgroup with <140 mmHg but not those with SBP ≥140 mmHg (p-for interaction 0.0060); however, the p_{interaction} was 0.1113 for SBP < versus ≥160 mmHg. Event-rates for hHF were increased by 2.7-fold in participants in the placebo groups with prevalent kidney disease (defined as eGFR <60 ml/min/1.73m² and macroalbuminuria: 3.65 per 100-patient-years vs 1.37 in those without) at baseline, and by 4.2-fold in participants with low eGFR (eGFR <30:

6.23 per 100-patient years vs 1.47 with eGFR ≥60), however, no differential effect by treatment arm was noted (p_{interaction}=0.3918, and 0.8827).

At baseline, LV EF was captured for 945 (13.5%) of participants within a year prior to randomization (458 in the linagliptin-and 487 in the placebo group). The mode of EF assessment varied, but echocardiography was by far the most commonly used method (90.2%) and average days between EF-assessments and randomization were 127 and 153 days, respectively in the linagliptin and placebo groups. The average pre randomization EF was 54% in the linagliptin group and 55% in the placebo group, with 31.9% and 29.2%, respectively, having EF ≤50% (mean LV EF respectively 39.1±8.4% and 39.2±7.6%), and only 11.6% and 11.7% having EF≤40% (mean LV EF respectively 29.7±6.4% and 31.7±6.1%). In total, 116 hHF events occurred in participants with EF-assessment prior to randomization. Among these with at least one hHF event, the average pre-randomization EF was 46.1±13.8% vs 47.7±12.8% in the linagliptin vs placebo group, respectively, whereas corresponding average pre-EF in those without a hFH event 54.7±11.8% and 55.2±12.0%. There was no heterogeneity of linagliptin effect on risk by pre-randomization EF categorized by EF < or ≥50% for hHF (p_{interaction}=0.141), for the composite outcome of hHF or CV death (p_{interaction}=0.158), or 3-point MACE (p_{interaction}=0.310).

### Other Safety and Adverse Events

Adverse events, serious adverse events, and adverse events leading to study drug discontinuation occurred in a similar proportion of patients treated with linagliptin or placebo (Table 6). Numerical imbalances for pemphigoid events (linagliptin 7 [0.2%] vs 0 placebo], skin lesions (linagliptin 5 [0.2%] vs placebo 1 [<0.1%)]), and adjudication-confirmed acute pancreatitis events (linagliptin 9 [0.3%] vs placebo 5 [0.1%]) were observed. Adjudication-confirmed events of chronic pancreatitis occurred with similar frequency (linagliptin 2 [0.1%] vs placebo 3 [0.1%]).

Malignancies occurred with similar frequency in both groups (linagliptin 116 [3.3%] vs placebo 134 [3.8%]). Overall events of reported pancreatic cancers were rare, but numerically higher in the linagliptin (11 [0.3%]) than the placebo group (4 [0.1%]). The oncology expert assessment committee deemed 1 case in each treatment arm to be possibly related to study drug treatment.

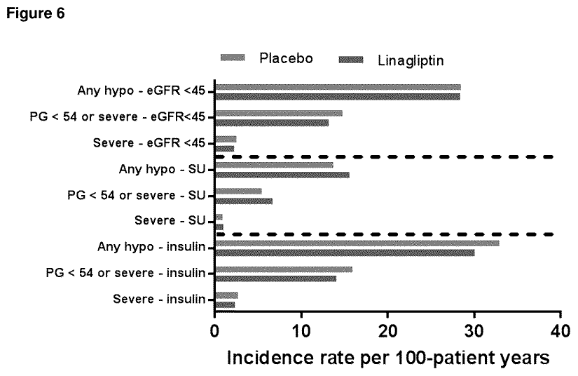

Confirmed hypoglycemic adverse events (including events of severe hypoglycemia) occurred in a similar proportion of patients in the linagliptin and placebo arms overall (Table 6, FIG. 3B). A numerically higher rate of hypoglycemia was observed with linagliptin compared to placebo in patients taking sulfonylurea at baseline, but not in other subgroups at increased risk for hypoglycemia (FIG. 6).

TABLE 5
Baseline characteristics

| | Linagliptin | Placebo | Total |
|---|---|---|---|
| | (n = 3494) | (n = 3485) | (n = 6979) |
| Age, years | 66.1 ± 9.05 | 65.6 ± 9.14 | 65.9 ± 9.10 |
| Male, n (%) | 2148 (61.5) | 2242 (64.3) | 4390 (62.9) |
| Race, n (%) | | | |
| White | 2827 (80.9) | 2769 (79.5) | 5596 (80.2) |
| Asian | 307 (8.8) | 333 (9.6) | 640 (9.2) |
| Black/African American | 194 (5.6) | 217 (6.2) | 411 (5.9) |
| Other^{¤} | 166 (4.8) | 166 (4.8) | 332 (4.8) |
| Region, n (%) | | | |
| Europe (incl South-Africa) | 1473 (42.2) | 1461 (41.9) | 2934 (42.0) |
| Latin America | 1156 (33.1) | 1154 (33.1) | 2310 (33.1) |
| North America | 593 (17.0) | 587 (16.8) | 1180 (16.9) |
| Asia | 272 (7.8) | 283 (8.1) | 555 (8.0) |
| Smoking status, n (%) | | | |
| Never smoker | 1897 (54.3) | 1856 (53.3) | 3753 (53.8) |
| Ex-smoker | 1231 (35.2) | 1276 (36.6) | 2507 (35.9) |
| Current smoker | 362 (10.4) | 350 (10.0) | 712 (10.2) |
| Missing | 4 (0.1) | 3 (0.1) | 7 (0.1) |
| History of heart failure, n (%) | 952 (27.2) | 921 (26.4) | 1873 (26.8) |
| Ischaemic heart disease, n (%) | 2029 (58.1) | 2052 (58.9) | 4081 (58.5) |
| History of hypertension, n (%) | 3171 (90.8) | 3178 (91.2) | 6349 (91.0) |
| Atrial fibrillation, n (%) | 319 (9.1) | 354 (10.2) | 673 (9.6) |
| eGFR (MDRD), mL/min/1.73 m² | 54.7 ± 25.09 | 54.5 ± 24.92 | 54.6 ± 25.00 |
| eGFR (MDRD), n (%) | | | |
| ≥90 mL/min/1.73 m² | 363 (10.4) | 365 (10.5) | 728 (10.4) |
| ≥60 ml/min/1.73 m² | 1294 (37.0) | 1337 (38.4) | 2631 (37.7) |
| ≥45-<60 ml/min/1.73 m² | 690 (19.7) | 658 (18.9) | 1348 (19.3) |
| ≥30-<45 ml/min/1.73 m² | 994 (28.4) | 944 (27.1) | 1938 (27.8) |
| <30 ml/min/1.73 m² | 516 (14.8) | 546 (15.7) | 1062 (15.2) |
| UACR, mg/g, median | 162 (43-700) | 162 (44-750) | 162 (44-728) |
| (25^{th}-75^{th} percentile) | | | |
| UACR, n (%)* | | | |
| <30 mg/g | 696 (20.0) | 696 (20.0) | 1392 (19.9) |
| 30-300 mg/g | 1463 (41.9) | 1431 (41.1) | 2894 (41.5) |
| >300 mg/g | 1333 (38.2) | 1357 (38.9) | 2690 (38.5) |
| BMI, kg/m² | 31.24 ± 5.29 | 31.31 ± 5.37 | 31.27 ± 5.33 |
| HbA1c, % | 7.94 ± 1.00 | 7.96 ± 1.01 | 7.95 ± 1.01 |
| Fasting plasma glucose, mg/dL | 151.2 ± 45.95 | 151.2 ± 45.95 | 151.2 ± 45.95 |
| Diabetes duration, years | 14.97 ± 9.64 | 14.53 ± 9.25 | 14.75 ± 9.45 |
| Systolic blood pressure, mmHg | 140.4 ± 17.7 | 140.6 ± 18.0 | 140.5 ± 17.9 |
| Diastolic blood pressure, mmHg | 77.8 ± 10.5 | 77.9 ± 10.4 | 77.8 ± 10.5 |
| Heart rate, bpm, mean ± SD | 69.8 ± 12.2 | 69.8 ± 12.3 | 69.8 ± 12.2 |
| Total cholesterol, mmol/L (mg/dL) | 4.5 ± 1.3 (173 ± 49) | 4.4 ± 1.2 (171 ± 47) | 4.5 ± 1.3 (172 ± 48) |
| LDL cholesterol, mmol/L (mg/dL) | 2.4 ± 1.0 (92 ± 40) | 2.3 ± 1.0 (91 ± 39) | 2.4 ± 1.0 (91 ± 40) |
| HDL cholesterol, mmol/L (mg/dL) | 1.2 ± 0.3 (45 ± 13) | 1.2 ± 0.3 (44 ± 13) | 1.2 ± 0.3 (45 ± 13) |
| Triglycerides, mmol/L (mg/dL) | 2.1 ± 1.5 (190 ± 136) | 2.1 ± 1.5 (187 ± 130) | 2.1 ± 1.5 (188 ± 133) |
| Glucose-lowering therapy, n (%) | | | 6802 (97.4) |
| Metformin | 1881 (53.8) | 1927 (55.3) | 3808 (54.6) |
| Sulfonylurea | 1102 (31.5) | 1140 (32.7) | 2224 (32.1) |
| Insulin | 2056 (58.8) | 1995 (57.2) | 4051 (58.0) |
| Antihypertensives, n (%) | | | |
| ACE inhibitors or ARBs | 2860 (81.9) | 2798 (80.3) | 5658 (81.1) |
| β-blockers | 2080 (59.5) | 2073 (59.5) | 4153 (59.5) |
| Diuretics | 1892 (54.1) | 1936 (55.6) | 3828 (54.9) |
| Calcium antagonists | 1433 (41.0) | 1446 (41.5) | 2879 (41.3) |
| Aspirin, n (%) | 2166 (62.0) | 2178 (62.5) | 4344 (62.2) |
| Statins, n (%) | 2495 (71.4) | 2523 (72.4) | 5018 (71.9) |

Data are mean ± SD unless otherwise specified.
^{¤}American Indian/Alaska Native or Native Hawaiian/other Pacific Islander
*UACR: Data missing for 3 (0.0%) patients: 2 (0.1%) linagliptin and 1 (0.0%) placebo.
ACE angiotensin-converting enzyme,
ARB angiotensin-receptor blocker,
BMI body-mass index,
eGFR estimated glomerular filtration rate,
HbA1c glycated hemoglobin A1c,
HDL high-density lipoprotein,
LDL low-density lipoprotein,
MDRD Modification of Diet in Renal Disease study equation,
UACR urinary albumin-to-creatinine ratio.

TABLE 6
Cardiovascular Outcomes, Kidney Outcomes, Adverse events and Hypoglycemic events

| Linagliptin | | Placebo | | | |
|---|---|---|---|---|---|
| (N = 3494) | | (N = 3485) | | | |
| | Rate/1000 | | Rate/1000 | Hazard ratio | |
| no. (%) | patient-years | no. (%) | patient-years | (95% Cl)* | p-value |

Cardiovascular, mortality and heart failure outcomes

| | | | | | | |
|---|---|---|---|---|---|---|
| Cardiovascular death, | 434 | 57.7 | 420 | 56.3 | 1.02 | |
| non-fatal myocardial | (12.4) | | (12.1) | | (0.89, 1.17) | |
| infarction, or non-fatal | | | 225 | | | |
| stroke (3-point MACE): | | | (6.5) | | | |
| primary outcome | | | 132 | | | |
| CV death | 221 | | (3.8) | | | |
| | (6.3) | | 63 | | | |
| Non-fata MI | 154 | | (1.8) | | | |
| | (4.4) | | | | | |
| Non-fatal stroke | 59 | | | | | |
| | (1.7) | | | | | |
| Non-inferiority | | | | | | 0.0002 |
| Superiority | | | | | | 0.7398 |
| All-cause death | 367 | 46.9 | 373 | 48.0 | 0.98 | 0.7402 |
| | (10.5) | | (10.7) | | (0.84, 1.13) | |
| Cardiovascular death | 255 | 32.6 | 264 | 34.0 | 0.96 | 0.6282 |
| | (7.3) | | (7.6) | | (0.81, 1.14) | |
| Non-cardiovascular | 112 | 14.3 | 109 | 14.0 | 1.02 | 0.8927 |
| death | (3.2) | | (3.1) | | (0.78, 1.33) | |
| Fatal myocardial | 11 | 1.4 | 14 | 1.8 | 0.78 | 0.5437 |
| infarction | (0.3) | | (0.4) | | (0.36, 1.72) | |
| Fatal or non-fatal | 165 | 21.8 | 146 | 19.4 | 1.12 | 0.3021 |
| myocardial infarction | (4.7) | | (4.2) | | (0.90, 1.40) | |
| Non-fatal myocardial | 156 | 20.6 | 135 | 18.0 | 1.15 | 0.2345 |
| infarction | (4.5) | | (3.9) | | (0.91, 1.45) | |
| Fatal stroke | 17 | 2.2 | 16 | 2.1 | 1.05 | 0.8779 |
| | (0.5) | | (0.5) | | (0.53, 2.09) | |
| Fatal or non-fatal | 81 | 10.6 | 88 | 11.6 | 0.91 | 0.5336 |
| stroke | (2.3) | | (2.5) | | (0.67, 1.23) | |
| Non-fatal stroke | 65 | 8.5 | 73 | 9.6 | 0.88 | 0.4495 |
| | (1.9) | | (2.1) | | (0.63, 1.23) | |
| 4-point MACE | 463 | 62.0 | 459 | 62.1 | 1.00 | 0.9598 |
| (3-point MACE or | (13.3) | | (13.2) | | (0.88, 1.13) | |
| hospitalization for | | | | | | |
| unstable angina) | | | | | | |
| Hospitalization for | 42 | 5.5 | 48 | 6.3 | 0.87 | 0.4956 |
| unstable angina | (1.2) | | (1.4) | | (0.57, 1.31) | |
| Coronary | 160 | 21.2 | 149 | 19.9 | 1.07 | 0.5727 |
| revascularization | (4.6) | | (4.3) | | (0.85, 1.33) | |
| procedure | | | | | | |
| Hospitalization for | 209 | 27.7 | 226 | 30.4 | 0.90 | 0.2635 |
| heart failure | (6.0) | | (6.5) | | (0.74, 1.08) | |
| Hospitalization for | 406 | 53.7 | 422 | 56.6 | 0.94 | 0.3881 |
| heart failure or | (11.6) | | (12.1) | | (0.82, 1.08) | |
| cardiovascular death | | | | | | |

Kidney outcomes

| | | | | | | |
|---|---|---|---|---|---|---|
| Renal death, sustained | 327 | 48.9 | 306 | 46.6 | 1.04 | 0.6164 |
| ESKD or sustained | (9.4) | | (8.8) | | (0.89, 1.22) | |
| decrease of 40% or | | | | | | |
| more in eGFR from | | | | | | |
| baseline (kidney | | | | | | |
| composite outcome): | | | | | | |
| key secondary | | | | | | |
| outcome | | | | | | |
| Renal death | | | | | | |
| ESKD | 1 | | 1 | | | |
| | (0.03) | | (0.03) | | | |
| Sustained decrease of | 63 | | 64 | | | |
| 40% or more in eGFR | (1.8) | | (1.8) | | | |
| Renal death, sustained | 263 | | 241 | | 0.98 | 0.871 |
| ESKD, or sustained | (7.5) | | (6.9) | | (0.82, 1.18) | |
| decrease of 50% or | | | | | | |
| more in eGFR from | | | | | | |
| baseline | | | | | | |
| Renal death or | 136 | 17.8 | 154 | 20.4 | 0.87 | 0.2371 |
| sustained ESKD | (3.9) | | (4.4) | | (0.69, 1.10) | |
| Albuminuria | 763 | 213.6 | 819 | 245.4 | 0.86 | 0.0034 |
| progression | (35.3) | | (38.5) | | (0.78, 0.95) | |
| Composite | 785 | 221.4 | 843 | 254.2 | 0.86 | 0.0032 |
| microvascular | (36.3) | | (39.6) | | (0.78, 0.95) | |
| endpoint* | | | | | | |
| Composite ocular | 36 | 4.7 | 49 | 6.5 | 0.73 | 0.1472 |
| endpoint¤ | (1.0) | | (1.4) | | (0.47, 1.12) | |

| Linagliptin | | Placebo | |
|---|---|---|---|
| (n = 3494) | | (n = 3485) | |
| N | % | N | % |

Adverse events

| | | | | |
|---|---|---|---|---|
| Any adverse events | 2697 | 77.2 | 2723 | 78.1 |
| Serious adverse | 1293 | 37.0 | 1343 | 38.5 |
| events | | | | |
| Adverse events | 359 | 10.3 | 402 | 11.5 |
| leading to | | | | |
| discontinuation | | | | |
| Hypersensitivity | 114 | 3.3 | 109 | 3.1 |
| reactions§, all AEs | | | | |
| Angioedema events | 13 | 0.45 | 16 | 0.57 |
| with concomitant | | | | |
| ACE/ARB use at | | | | |
| baseline | | | | |
| Pemphigoid | 7 | 0.2 | 0 | 0.0 |
| Skin lesions | 5 | 0.1 | 1 | <0.1 |
| Acute pancreatitis, | 9¹ | 0.3 | 5 | 0.1 |
| adjudication confirmed | | | | |
| Chronic pancreatitis, | 2 | 0.1 | 3 | 0.1 |
| adjudication confirmed | | | | |
| All Cancers | 116 | 3.3 | 134 | 3.8 |
| Colon Cancer | 6 | 0.2 | 8 | 0.2 |
| Pancreatic | 11 | 0.3 | 4 | 0.1 |
| Cancer² | | | | |
| Gastric Cancer | 0 | 0.0 | 3 | 0.1 |

Hypoglycemic events

| | | | | |
|---|---|---|---|---|
| Investigator reported | 1036 | 29.7 | 1024 | 29.4 |
| hypoglycemia | | | | |
| Confirmed | 557 | 15.9 | 572 | 16.4 |
| hypoglycemic | | | | |
| adverse events with | | | | |
| plasma glucose <54 | | | | |
| mg/dl or severe | | | | |
| event^{‡*} | | | | |
| Severe event^{‡} | 106 | 3.0 | 108 | 3.1 |

HR based on Cox regression analyses in patients treated with ≥1 dose of study drug.
*Time to first renal death, ESKD, sustained decrease of >50% in eGFR, albuminuria progression, retinal photocoagulation or anti-VEGF injection therapy for diabetic retinopathy, vitreous haemorrhage, diabetes related blindness.
¤Time to first use of retinal laser coagulation therapy or treatment with intravitreal injection(s) of an anti-VEGF therapy for diabetic retinopathy or vitreous haemorrhage, or diabetes-related blindness.
Adverse events is classified based on MedDRA version 20.1 and include AEs from patients treated with ≥1 dose of study drug until ≤7 days after the last Intake of study medication with the exception of pancreatitis and cancers that include all events in patients treated with ≥1 dose of study drug until study end.
¹n = 2 (0.1%) fatal cases of pancreatitis
²adjudication confirmed
^{§}Based on 276 MedDRA 20.1 preferred terms
^{‡}Requiring the assistance of another person to actively administer carbohydrate, glucagon or other resuscitative actions.

TABLE 7
Hazard ratios for the primary outcome (3-point MACE) in subgroups

| | Patients with event/ | | | |
|---|---|---|---|---|
| | patients analyzed | | Hazard | |
| | Linagliptin | Placebo | ratio | (95% Cl) |
| All patients | 434/3493 | 420/3485 | 1.02 | 0.89, 1.17 |
| Age^{a} | | | | |
| <65 years | 154/1467 | 140/1501 | 1.11 | 0.89, 1.40 |
| ≥65 years | 280/2027 | 280/1984 | 0.97 | 0.82, 1.15 |
| Gender | | | | |
| Male | 282/2148 | 276/2242 | 1.06 | 0.90, 1.25 |
| Female | 152/1346 | 144/1243 | 0.96 | 0.77, 1.21 |
| Race | | | | |
| White | 340/2827 | 341/2769 | 0.97 | 0.83, 1.13 |
| Asian | 40/307 | 40/333 | 1.09 | 0.70, 1.70 |
| Black/African-American | 31/194 | 27/217 | 1.30 | 0.78, 2.18 |
| Other | 23/166 | 12/166 | 1.86 | 0.93, 3.75 |
| Ethnicity | | | | |
| Hispanic/Latino | 143/1227 | 130/1274 | 1.13 | 0.89, 1.43 |
| Not Hispanic/Latino | 291/2267 | 290/2211 | 0.97 | 0.83, 1.14 |
| Region^{b} | | | | |
| Europe + South Africa | 182/1473 | 196/1461 | 0.92 | 0.75, 1.12 |
| North America | 91/593 | 72/587 | 1.25 | 0.92, 1.71 |
| Latin America | 132/1156 | 119/1154 | 1.10 | 0.86, 1.40 |
| Asia | 29/272 | 33/283 | 0.90 | 0.55, 1.48 |
| Glycated hemoglobin* | | | | |
| <8.0% | 229/1915 | 243/1855 | 0.90 | 0.75, 1.08 |
| ≥8.0% | 205/1579 | 177/1630 | 1.20 | 0.98, 1.46 |
| Body mass index | | | | |
| <30 kg/m² | 191/1516 | 189/1517 | 0.98 | 0.80, 1.20 |
| ≥30 kg/m² | 243/1978 | 230/1965 | 1.06 | 0.89, 1.27 |
| Blood pressure control^{c} | | | | |
| SBP ≥140 mmHg or | 249/1800 | 231/1834 | 1.11 | 0.93, 1.33 |
| DBP ≥90 mmHg | | | | |
| SBP <140 mmHg and | 185/1694 | 189/1651 | 0.93 | 0.76, 1.14 |
| DBP <90 mmHg | | | | |
| Estimated glomerular | | | | |
| filtration rate^{d} | | | | |
| ≥60 mL/min/1.73 m² | 103/1294 | 110/1337 | 0.96 | 0.73, 1.25 |
| ≥45 to <60 mL/min/1.73 m² | 81/690 | 69/658 | 1.12 | 0.81, 1.54 |
| ≥30 to <45 mL/min/1.73 m² | 149/994 | 133/944 | 1.07 | 0.84, 1.35 |
| <30 mL/min/1.73 m² | 101/516 | 108/546 | 0.97 | 0.74, 1.27 |
| Urine albumin-to- | | | | |
| creatinine ratio | | | | |
| <30 mg/g | 67/696 | 60/696 | 1.10 | 0.78, 1.56 |
| 30 to 300 mg/g | 158/1463 | 160/1431 | 0.95 | 0.77, 1.19 |
| >300 mg/g | 208/1333 | 199/1357 | 1.06 | 0.88, 1.29 |
| Metformin | | | | |
| No | 242/1613 | 230/1558 | 1.02 | 0.85, 1.22 |
| Yes | 192/1881 | 190/1927 | 1.02 | 0.83, 1.25 |
| Metformin-dose | | | | |
| ≤1500 mg | 81/787 | 80/792 | 1.02 | 0.75, 1.39 |
| >1500 mg | 111/1094 | 110/1135 | 1.02 | 0.78, 1.33 |
| Not on metformin | 242/1613 | 230/1558 | 1.02 | 0.85, 1.22 |
| Sulfonylurea | | | | |
| No | 315/2392 | 314/2345 | 0.98 | 0.84, 1.14 |
| Yes | 119/1102 | 106/1140 | 1.15 | 0.88, 1.49 |
| Insulin | | | | |
| No | 139/1487 | 159/1542 | 0.88 | 0.70, 1.11 |
| Yes | 295/2007 | 261/1943 | 1.10 | 0.93, 1.30 |
| Lipid lowering drugs | | | | |
| No | 95/871 | 99/839 | 0.90 | 0.68, 1.20 |
| Yes | 339/2623 | 321/2646 | 1.06 | 0.91, 1.24 |
| Angiotensin-converting | | | | |
| enzyme inhibitors/ | | | | |
| angiotensin receptor | | | | |
| blockers | | | | |
| No | 89/634 | 101/687 | 0.93 | 0.70, 1.23 |
| Yes | 345/2860 | 319/2798 | 1.06 | 0.91, 1.23 |
| Calcium channel | | | | |
| blockers (CCB)* | | | | |
| No | 239/2061 | 256/2039 | 0.91 | 0.76, 1.08 |
| Yes | 195/1433 | 164/1446 | 1.21 | 0.98, 1.49 |
| Beta blockers | | | | |
| No | 134/1414 | 152/1412 | 0.87 | 0.69, 1.09 |
| Yes | 300/2080 | 268/2073 | 1.11 | 0.95, 1.31 |
| Diuretics | | | | |
| No | 159/1602 | 134/1549 | 1.15 | 0.92, 1.45 |
| Yes | 275/1892 | 286/1936 | 0.97 | 0.82, 1.14 |
| Antiplatelet drugs | | | | |
| No | 125/1102 | 115/1084 | 1.10 | 0.85, 1.42 |
| Yes | 309/2392 | 305/2401 | 0.99 | 0.85, 1.16 |
| History of heart failure | | | | |
| No | 275/2542 | 269/2564 | 1.02 | 0.86, 1.21 |
| Yes | 159/952 | 151/921 | 1.01 | 0.81, 1.27 |
| Duration of type 2 diabetes | | | | |
| ≤5 years | 45/521 | 47/553 | 0.98 | 0.65, 1.48 |
| >5 to <10 years | 73/696 | 71/688 | 1.01 | 0.73, 1.40 |
| ≥10 years | 316/2277 | 302/2244 | 1.03 | 0.88, 1.20 |
| CKD prognosis by KDIGO^{e} | | | | |
| Low | 11/232 | 11/252 | 1.13 | 0.49, 2.60 |
| Medium | 61/766 | 68/795 | 0.89 | 0.63, 1.26 |
| High | 111/995 | 96/905 | 1.05 | 0.80, 1.38 |
| Very high | 250/1499 | 245/1533 | 1.04 | 0.87, 1.24 |
| Cardiorenal risk by | | | | |
| combinations of | | | | |
| macrovascular disease, | | | | |
| albuminuria and eGFR^{f} | | | | |
| Cat A | 117/1361 | 120/1367 | 0.97 | 0.75, 1.25 |
| Cat B | 86/394 | 75/345 | 0.92 | 0.67, 1.25 |
| Cat C | 33/253 | 44/270 | 0.76 | 0.49, 1.20 |
| Cat D | 163/1153 | 147/1156 | 1.12 | 0.89, 1.40 |
| Cat E | 32/309 | 30/303 | 1.13 | 0.68, 1.85 |
| Cardiorenal risk | | | | |
| Albuminuria and previous | 117/1361 | 120/1367 | 0.97 | 0.75, 1.25 |
| macrovascular disease | | | | |
| without eGFR >45 | | | | |
| mL/min/1.73 m2 | | | | |
| Albuminuria and previous | 195/1462 | 177/1459 | 1.12 | 0.91, 1.37 |
| macrovascular disease plus | | | | |
| renal impairment (eGFR | | | | |
| 15-<45 mL/min/1.73 m² | | | | |
| with any UACR mg/g) | | | | |
| Albuminuria and previous | 119/647 | 119/615 | 0.88 | 0.69, 1.14 |
| macrovascular disease plus | | | | |
| eGFR 45-75 mL/min/ | | | | |
| 1.73 m² with an | | | | |
| UACR >200 mg/g | | | | |
| Established renal disease^{g} | | | | |
| Yes | 314/2109 | 296/2074 | 1.04 | 0.89, 1.22 |
| No | 120/1385 | 124/1411 | 0.98 | 0.76, 1.25 |
| Established macrovascular | | | | |
| disease and albuminuria | | | | |
| Yes | 236/2008 | 239/1982 | 0.94 | 0.79, 1.13 |
| No | 198/1486 | 181/1503 | 1.13 | 0.92, 1.38 |
| Prevalent kidney disease | | | | |
| (eGFR<60 mL/min/1.73 m² | | | | |
| or macroalbuminuria | | | | |
| UACR >300 mg/g) | | | | |
| Yes | 374/2606 | 348/2541 | 1.04 | 0.90, 1.21 |
| No | 60/887 | 72/944 | 0.88 | 0.62, 1.24 |

Cox regression analysis in patients treated with ≥1 dose of study drug. Subgroup factors were pre-specified for the primary outcome.
*p <0.05 for the test of homogeneity of the treatment group difference among subgroups (test for group by covariate interaction) with no adjustment for multiple tests;
p = 0.0403 for CCB, p = 0.0407 for glycated hemoglobin.
^{a}consistent results in the additional prespecifed age subgroups <65, 65-75 and >75 years,
^{b}an additional prespecifed regional subgroup analyses (Japan, non-Japan) involved too few events to be analysed,
^{c}consistent results in the additional prespecifed BP subgroups: SBP <140 and >=140 mmHg and <160 and >=160 mmHg,
^{d}consistent results in the additional prespecifed eGFR subgroups <60 and >=60 ml/min/1.73 m²,
^{e}Per 2012 KDIGO criteria; Low risk defined as eGFR ≥60 ml/min/1.73 m² and UACR <30 mg/g, Moderately increased risk defined as eGFR 45-59 ml/min/1.73 m² and UACR <30 mg/g, or eGFR ≥60 ml/min/1.73 m² and UACR 30-300 mg/g, High risk defined as eGFR 30-44 ml/min/1.73 m² and UACR <30 mg/g, eGFR 45-59 ml/min/1.73 m² and UACR 30-300 mg/g, or eGFR ≥60 and UACR >300 mg/g, Very high risk defined as eGFR <30 ml/min/1.73 m² with any UACR, eGFR 30-44 and UACR 30-300 mg/g, or eGFR 45-59 ml/min/1.73 m² and UACR >300 mg/g,
^{f}A) albuminuria and previous macrovascular disease without evidence of impaired renal function, B) albuminuria and previous macrovascular disease plus renal impairment (eGFR 15-<45 mL/min/1.73 m² with any UACR mg/g), C) albuminuria and previous macrovascular disease plus renal impairment (eGFR ≥45-75 mL/min/1.73 m² with an UACR >200 mg/g), D) impaired renal function (eGFR 15-<45 mL/min/1.73 m² with any UACR), E) impaired Renal function (eGFR ≥45-75 mL/min/1.73 m² with an UACR >200 mg/g),
^{g}patients in the “yes” category fulfils any one of the categories: albuminuria and previous macrovascular disease plus renal impairment (eGFR 15-<45mL/min/1.73 m² with any UACR), albuminuria and previous macrovascular disease plus renal impairment (eGFR 45-75 mL/min/1.73 m² with an UACR >200 mg/g), impaired renal function (eGFR 15-<45 mL/min/1.73 m² with any UACR mg/g), impaired renal function (eGFR 45-75 mL/min/1.73 m² with UACR >200 mg/g).

TABLE 8
Hazard ratios for the key secondary kidney outcome in subgroups

| | Patients with event/ | | | |
|---|---|---|---|---|
| | patients analyzed | | Hazard | |
| | Linagliptin | Placebo | ratio | (95% Cl) |
| All patients | 327/3493 | 306/3485 | 1.04 | 0.89, 1.22 |
| Age^{a} | | | | |
| <65 years | 180/1467 | 173/1501 | 1.05 | 0.85, 1.29 |
| ≥65 years | 147/2027 | 133/1984 | 1.05 | 0.83, 1.33 |
| Gender | | | | |
| Male | 210/2148 | 189/2242 | 1.12 | 0.92, 1.36 |
| Female | 117/1346 | 117/1243 | 0.92 | 0.71, 1.19 |
| Race | | | | |
| White | 237/2827 | 220/2769 | 1.03 | 0.86, 1.24 |
| Asian | 32/307 | 37/333 | 0.90 | 0.56, 1.44 |
| Black/African-American | 35/194 | 30/217 | 1.31 | 0.80, 2.13 |
| Other | 23/166 | 19/166 | 1.16 | 0.63, 2.14 |
| Ethnicity | | | | |
| Hispanic/Latino | 156/1227 | 142/1274 | 1.10 | 0.88, 1.38 |
| Not Hispanic/Latino | 171/2267 | 164/2211 | 0.99 | 0.80, 1.23 |
| Region^{b} | | | | |
| Europe + South Africa | 98/1473 | 98/1461 | 0.96 | 0.72, 1.27 |
| North America | 51/593 | 43/587 | 1.19 | 0.79, 1.78 |
| Latin America | 149/1156 | 134/1154 | 1.07 | 0.85, 1.36 |
| Asia | 29/272 | 31/283 | 0.96 | 0.58, 1.59 |
| Glycated hemoglobin* | | | | |
| <8.0% | 186/1915 | 158/1855 | 1.13 | 0.91, 1.40 |
| ≥8.0% | 141/1579 | 148/1630 | 0.94 | 0.75, 1.19 |
| Body mass index | | | | |
| <30 kg/m² | 162/1516 | 135/1517 | 1.14 | 0.91, 1.43 |
| ≥30 kg/m² | 165/1978 | 171/1965 | 0.96 | 0.77, 1.19 |
| Blood pressure control^{c} | | | | |
| SBP 140 mmHg or | 222/1800 | 205/1834 | 1.08 | 0.89, 1.31 |
| DBP ≥90 mmHg | | | | |
| SBP <140 mmHg and | 105/1694 | 101/1651 | 0.99 | 0.75, 1.30 |
| DBP <90 mmHg | | | | |
| Estimated glomerular | | | | |
| filtration rate^{d} | | | | |
| ≥60 mL/min/1.73 m² | 54/1294 | 38/1337 | 1.46 | 0.97, 2.21 |
| ≥45 to <60 mL/min/1.73 m² | 51/690 | 49/658 | 0.94 | 0.64, 1.39 |
| ≥30 to <45 mL/min/1.73 m² | 89/994 | 86/944 | 0.95 | 0.70, 1.27 |
| <30 mL/min/1.73 m² | 133/516 | 133/546 | 1.05 | 0.82, 1.33 |
| Urine albumin-to- | | | | |
| creatinine ratio | | | | |
| <30 mg/g | 22/696 | 16/696 | 1.46 | 0.77, 2.79 |
| 30 to 300 mg/g | 53/1463 | 38/1431 | 1.30 | 0.86, 1.98 |
| >300 mg/g | 252/1333 | 251/1357 | 0.97 | 0.81, 1.15 |
| Metformin | | | | |
| No | 212/1613 | 203/1558 | 0.99 | 0.82, 1.20 |
| Yes | 115/1881 | 103/1927 | 1.11 | 0.85, 1.44 |
| Metformin-dose | | | | |
| ≤1500 mg | 53/787 | 39/792 | 1.29 | 0.85, 1.95 |
| >1500 mg | 62/1094 | 64/1135 | 0.99 | 0.70, 1.40 |
| Not on metformin | 212/1613 | 203/1558 | 0.99 | 0.82, 1.20 |
| Sulfonylurea | | | | |
| No | 252/2392 | 220/2345 | 1.10 | 0.92, 1.32 |
| Yes | 75/1102 | 86/1140 | 0.87 | 0.64, 1.19 |
| Insulin | | | | |
| No | 101/1487 | 94/1542 | 1.08 | 0.82, 1.43 |
| Yes | 226/2007 | 212/1943 | 1.01 | 0.84, 1.22 |
| Lipid lowering drugs | | | | |
| No | 101/871 | 82/839 | 1.13 | 0.85, 1.52 |
| Yes | 226/2623 | 224/2646 | 1.00 | 0.83, 1.20 |
| Angiotensin-converting | | | | |
| enzyme inhibitors/ | | | | |
| angiotensin receptor | | | | |
| blockers | | | | |
| No | 62/634 | 69/687 | 0.96 | 0.68, 1.36 |
| Yes | 265/2860 | 237/2798 | 1.07 | 0.89, 1.27 |
| Calcium channel | | | | |
| blockers (CCB) | | | | |
| No | 155/2061 | 147/2039 | 1.03 | 0.82, 1.29 |
| Yes | 172/2860 | 159/2798 | 1.05 | 0.85, 1.31 |
| Beta blockers | | | | |
| No | 161/1414 | 142/1412 | 1.14 | 0.91, 1.42 |
| Yes | 166/2080 | 164/2073 | 0.97 | 0.78, 1.20 |
| Diuretics | | | | |
| No | 129/1602 | 117/1549 | 1.06 | 0.82, 1.36 |
| Yes | 198/1892 | 189/1936 | 1.04 | 0.85, 1.26 |
| Antiplatelet drugs | | | | |
| No | 131/1102 | 102/1084 | 1.25 | 0.97, 1.62 |
| Yes | 196/2392 | 204/2401 | 0.94 | 0.77, 1.14 |
| History of heart failure | | | | |
| No | 252/2542 | 230/2564 | 1.07 | 0.90, 1.28 |
| Yes | 75/952 | 76/921 | 0.95 | 0.69, 1.31 |
| Duration of type 2 diabetes* | | | | |
| ≤5 years | 41/521 | 22/553 | 1.97 | 1.17, 3.30 |
| >5 to <10 years | 56/699 | 55/688 | 0.94 | 0.65, 1.37 |
| ≥10 years | 230/2277 | 229/2244 | 0.97 | 0.81, 1.17 |
| CKD prognosis by KDIGO^{e} | | | | |
| Low | 8/232 | 2/252 | NC** | NC** |
| Medium | 14/766 | 17/795 | NC** | NC** |
| High | 57/995 | 37/905 | NC** | NC** |
| Very high | 248/1499 | 250/1533 | NC** | NC** |
| Cardiorenal risk by | | | | |
| combinations of | | | | |
| macrovascular disease, | | | | |
| albuminuria and eGFR^{f} | | | | |
| Cat A | 38/1361 | 31/1367 | 1.22 | 0.76, 1.96 |
| Cat B | 51/394 | 50/345 | 0.79 | 0.53, 1.16 |
| Cat C | 23/253 | 15/270 | 1.53 | 0.80, 2.94 |
| Cat D | 180/1153 | 176/1156 | 1.01 | 0.82, 1.24 |
| Cat E | 35/309 | 33/303 | 1.04 | 0.64, 1.67 |
| Cardiorenal risk | | | | |
| Albuminuria and previous | 38/1361 | 31/1367 | 1.22 | 0.76, 1.95 |
| macrovascular disease | | | | |
| without eGFR >45 | | | | |
| mL/min/1.73 m2 | | | | |
| Albuminuria and previous | 215/1462 | 209/1459 | 1.01 | 0.84, 1.23 |
| macrovascular disease plus | | | | |
| renal impairment (eGFR | | | | |
| 15-<45 mL/min/1.73 m² | | | | |
| with any UACR mg/g) | | | | |
| Albuminuria and previous | 74/647 | 65/615 | 0.99 | 0.71, 1.38 |
| macrovascular disease plus | | | | |
| eGFR 45-75 mL/min/ | | | | |
| 1.73 m² with an | | | | |
| UACR >200 mg/g | | | | |
| Established renal disease^{g} | | | | |
| Yes | 289/2109 | 274/2074 | 1.00 | 0.85, 1.18 |
| No | 38/1385 | 32/1411 | 1.20 | 0.75, 1.91 |
| Established macrovascular | | | | |
| disease and albuminuria | | | | |
| Yes | 112/2008 | 96/1982 | 1.11 | 0.85, 1.46 |
| No | 215/1486 | 210/1503 | 1.03 | 0.85, 1.24 |
| Prevalent kidney disease | | | | |
| (eGFR <60 mL/min/1.73 m² | | | | |
| or macroalbuminuria | | | | |
| UACR >300 mg/g) | | | | |
| Yes | 308/2606 | 291/2541 | 0.99 | 0.85, 1.17 |
| No | 19/887 | 15/944 | 1.36 | 0.69, 2.67 |

Cox regression analysis in patients treated with ≥1 dose of study drug. Subgroup factors were pre-specified for the primary outcome.
*p <0.05 for the test of homogeneity of the treatment group difference among subgroups (test for group by covariate interaction) with no adjustment for multiple tests;
p = 0.0377 for duration of type 2 diabetes.
**NC-not calculated owing to few events in some subgroups (<14).
^{a}consistent results in the additional prespecifed age subgroups <65, 65-75 and >75 years,
^{b}an additional prespecifed regional subgroup analyses (Japan, non-Japan) involved too few events to be analysed,
^{c}consistent results in the additional prespecifed BP subgroups: SBP <140 and >=140 mmHg and <160 and >=160 mmHg,
^{d}consistent results in the additional prespecifed eGFR subgroups <60 and >=60 ml/min/1.73 m2,
^{e}Per 2012 KDIGO criteria; Low risk defined as eGFR ≥60 ml/min/1.73 m² and UACR <30 mg/g, Moderately increased risk defined as eGFR 45-59 ml/min/1.73 m² and UACR <30 mg/g, or eGFR ≥60 ml/min/1.73 m² and UACR 30-300 mg/g,High risk defined as eGFR 30-44 ml/min/1.73 m² and UACR <30 mg/g, eGFR 45-59 ml/min/1.73 m² and UACR 30-300 mg/g, or eGFR ≥60 and UACR >300 mg/g, Very high risk defined as eGFR <30 ml/min/1.73 m² with any UACR, eGFR 30-44 and UACR 30-300 mg/g, or eGFR 45-59 ml/min/1.73 m² and UACR >300 mg/g,
^{f}A) albuminuria and previous macrovascular disease without evidence of impaired renal function, B) albuminuria and previous macrovascular disease plus renal impairment (eGFR 15-<45 mL/min/1.73 m² with any UACR mg/g), C) albuminuria and previous macrovascular disease plus renal impairment (eGFR ≥45-75 mL/min/1.73 m² with an UACR >200 mg/g), D) impaired renal function (eGFR 15-<45 mL/min/1.73 m² with any UACR), E) impaired Renal function (eGFR ≥45-75 mUmin/1.73 m² with an UACR >200 mg/g),
^{g}patients in the “yes” category fulfils any one of the categories: albuminuria and previous macrovascular disease plus renal impairment (eGFR 15-<45mL/min/1.73 m² with any UACR), albuminuria and previous macrovascular disease plus renal impairment (eGFR 45-75 mL/min/1.73 m² with an UACR >200 mg/g), impaired renal function (eGFR 15-<45 mL/min/1.73 m² with any UACR mg/g), impaired renal function (eGFR 45-75 mL/min/1.73 m² with UACR >200 mg/g).

TABLE 9
Distribution of events contributing to
the composite microvascular outcome

| | Linagliptin | Placebo |
|---|---|---|
| | (n = 3494) (%) | (n = 3485) (%) |
| Number of patients | 785 (22.5) | 843 (24.2) |

Kidney components

| | | |
|---|---|---|
| Patients with renal death | 0 | 0 |
| Patients with sustained ESKD | 10 (0.3) | 8 (0.2) |
| Patients with sustained >50% | 21 (0.6) | 14 (0.4) |
| eGFR decrease | | |
| Patients with albuminuria | 745 (21.3) | 810 (23.2) |
| progression | | |

Ocular components

| | | |
|---|---|---|
| Patients with retinal laser | 8 (0.2) | 9 (0.3) |
| coagulation or anti-VEGF | | |
| injection for diabetic | | |
| retinopathy | | |
| Patients with vitreous | 4 (0.1) | 5 (0.1) |
| haemorrhage | | |
| Patients with diabetes related | 0 | 0 |
| blindness | | |

TABLE 10
Distribution of events contributing to the composite ocular outcome

| | Linagliptin | Placebo |
|---|---|---|
| | (n = 3494) (%) | (n = 3485) (%) |
| Number of patients | 36 (1.0) | 49 (1.4) |
| Patients with retinal laser | 7 (0.2) | 11 (0.3) |
| coagulation | | |
| Patients with anti-VEGF | 10 (0.3) | 11 (0.3) |
| injection for diabetic | | |
| retinopathy | | |
| Patients with vitreous | 18 (0.5) | 27 (0.8) |
| haemorrhage | | |
| Patients with diabetes related | 2 (0.1) | 2 (0.1) |
| blindness | | |

TABLE 11
Hazard ratios for hospitalised heart failure in subgroups

| | Patients with event/ | | | | |
|---|---|---|---|---|---|
| | patients analysed | | Hazard | | p-for |
| | Linagliptin | Placebo | ratio | (95% CI) | interaction |
| All patients | 209/3494 | 226/3485 | 0.90 | 0.74, 1.08 | |
| Age^{a} | | | | | |
| <65 years | 67/1467 | 77/1501 | 0.87 | 0.63, 1.21 | 0.8504 |
| ≥65 years | 142/2027 | 149/1984 | 0.91 | 0.72, 1.14 | |
| Gender | | | | | |
| Male | 135/2148 | 157/2242 | 0.87 | 0.69, 1.10 | 0.6169 |
| Female | 74/1346 | 69/1243 | 0.97 | 0.70, 1.34 | |
| Race | | | | | |
| White | 174/2827 | 171/2769 | NC | | 0.2520 |
| Asian | 14/307 | 28/333 | | | |
| Black/African-American | 15/194 | 21/217 | | | |
| Other | 6/166 | 7/166 | | | |
| Ethnicity | | | | | |
| Hispanic/Latino | 58/1227 | 62/1274 | 0.96 | 0.67, 1.38 | 0.6334 |
| Not Hispanic/Latino | 151/2267 | 164/2211 | 0.87 | 0.70, 1.08 | |
| Region^{b} | | | | | |
| Europe + South Africa | 101/1473 | 88/1461 | 1.13 | 0.85, 1.51 | 0.0368 |
| North America | 42/593 | 61/587 | 0.65 | 0.44, 0.97 | |
| Latin America | 54/1156 | 54/1154 | 0.99 | 0.68, 1.44 | |
| Asia | 12/272 | 23/283 | 0.47 | 0.24, 0.95 | |
| Glycated haemoglobin | | | | | |
| <8.0% | 107/1915 | 126/1855 | 0.78 | 0.60, 1.01 | 0.1224 |
| ≥8.0% | 102/1579 | 100/1630 | 1.05 | 0.80, 1.39 | |
| Body mass index | | | | | |
| <30 kg/m² | 78/1516 | 79/1517 | 0.98 | 0.72, 1.34 | 0.5034 |
| ≥30 kg/m² | 131/1978 | 146/1965 | 0.86 | 0.68, 1.09 | |
| Blood pressure control^{c} | | | | | |
| SBP <140 mmHg | 84/1750 | 116/1701 | 0.68 | 0.51, 0.90 | 0.0068 |
| SBP ≥140 mmHg | 125/1744 | 110/1784 | 1.15 | 0.89, 1.48 | |
| SBP <160 mmHg | 164/3017 | 190/3020 | 0.84 | 0.68, 1.03 | 0.1113 |
| SBP ≥160 mmHg | 45/477 | 36/465 | 1.24 | 0.80, 1.92 | |
| Estimated glomerular | | | | | |
| filtration rate^{d} | | | | | |
| ≥60 mL/min/1.73 m² | 36/1294 | 41/1337 | 0.88 | 0.56, 1.37 | 0.8827 |
| ≥45 to <60 mL/min/1.73 m² | 38/690 | 32/658 | 1.06 | 0.66, 1.70 | |
| ≥30 to <45 mL/min/1.73 m² | 76/994 | 85/944 | 0.85 | 0.62, 1.16 | |
| <30 mL/min/1.73 m² | 59/516 | 68/546 | 0.94 | 0.66, 1.70 | |
| Urine albumin-to- | | | | | |
| creatinine ratio | | | | | |
| <30 mg/g | 26/696 | 32/696 | 0.76 | 0.45, 1.28 | 0.6157 |
| 30 to 300 mg/g | 72/1463 | 80/1431 | 0.86 | 0.63, 1.18 | |
| >300 mg/g | 111/1333 | 113/1357 | 0.99 | 0.76, 1.29 | |
| Metformin | | | | | |
| No | 138/1613 | 140/1558 | 0.97 | 0.77, 1.23 | 0.3213 |
| Yes | 71/1881 | 86/1927 | 0.79 | 0.58, 1.09 | |
| Metformin-dose | | | | | |
| ≤1500 mg | 22/787 | 32/792 | 0.65 | 0.38, 1.12 | 0.4180 |
| >1500 mg | 49/1094 | 54/1135 | 0.88 | 0.60, 1.30 | |
| Not on metformin | 138/1613 | 140/1558 | 0.97 | 0.77, 1.23 | |
| Sulfonylurea | | | | | |
| No | 166/2392 | 174/2345 | 0.92 | 0.74, 1.13 | 0.6398 |
| Yes | 43/1102 | 52/1140 | 0.82 | 0.55, 1.23 | |
| Insulin | | | | | |
| No | 40/1487 | 63/1542 | 0.62 | 0.42, 0.92 | 0.0360 |
| Yes | 169/2007 | 163/1943 | 1.00 | 0.81, 1.24 | |
| Lipid lowering drugs | | | | | |
| No | 34/871 | 32/839 | 1.01 | 0.62, 1.63 | 0.6196 |
| Yes | 175/2623 | 194/2646 | 0.88 | 0.72, 1.08 | |
| Angiotensin-converting | | | | | |
| enzyme inhibitors/ | | | | | |
| angiotensin receptor | | | | | |
| blockers | | | | | |
| No | 48/634 | 58/687 | 0.84 | 0.57, 1.24 | 0.6913 |
| Yes | 161/2860 | 168/2798 | 0.92 | 0.74, 1.14 | |
| Angiotensin-converting | | | | | |
| enzyme inhibitors | | | | | |
| No | 132/1920 | 130/1923 | 1.02 | 0.80, 1.29 | 0.1295 |
| Yes | 77/1574 | 96/1562 | 0.75 | 0.56, 1.02 | |
| Calcium channel | | | | | |
| blockers (CCB) | | | | | |
| No | 113/2061 | 126/2039 | 0.86 | 0.67, 1.11 | 0.6531 |
| Yes | 96/1433 | 100/1446 | 0.94 | 0.71, 1.25 | |
| Beta blockers | | | | | |
| No | 55/1414 | 58/1412 | 0.95 | 0.66, 1.38 | 0.7039 |
| Yes | 154/2080 | 168/2073 | 0.88 | 0.70, 1.09 | |
| Diuretics | | | | | |
| No | 53/1602 | 40/1549 | 1.26 | 0.84, 1.90 | 0.0746 |
| Yes | 156/1892 | 186/1936 | 0.83 | 0.67, 1.02 | |
| Loop diuretics | | | | | |
| No | 80/2530 | 79/2461 | 0.97 | 0.71, 1.32 | 0.7242 |
| Yes | 129/964 | 147/1024 | 0.90 | 0.71, 1.14 | |
| Antiplatelet drugs | | | | | |
| No | 61/1102 | 62/1084 | 0.94 | 0.66, 1.34 | 0.7805 |
| Yes | 148/2392 | 164/2401 | 0.88 | 0.71, 1.10 | |
| History of heart failure | | | | | |
| No | 96/2542 | 104/2564 | 0.92 | 0.70, 1.22 | 0.8104 |
| Yes | 113/952 | 122/921 | 0.88 | 0.68, 1.14 | |
| Atrial fibrillation | | | | | |
| No | 154/3175 | 173/3131 | 0.86 | 0.69, 1.07 | 0.3420 |
| Yes | 55/319 | 53/354 | 1.06 | 0.73, 1.55 | |
| Ischemic heart disease | | | | | |
| No | 66/1465 | 59/1433 | 1.04 | 0.73, 1.48 | 0.3493 |
| Yes | 143/2029 | 167/2052 | 0.85 | 0.68, 1.07 | |
| Duration of type 2 diabetes | | | | | |
| ≤5 years | 18/521 | 23/553 | 0.82 | 0.44, 1.52 | 0.9047 |
| >5 to <10 years | 42/696 | 41/688 | 0.96 | 0.63, 1.48 | |
| ≥10 years | 149/2277 | 162/2244 | 0.89 | 0.71, 1.11 | |
| CKD prognosis by KDIGO^{e} | | | | | |
| Low | 2/232 | 2/252 | NC | | 0.5572 |
| Medium | 18/766 | 29/795 | | | |
| High | 45/995 | 41/905 | | | |
| Very high | 144/1499 | 154/1533 | | | |
| Cardiorenal risk | | | | | |
| Albuminuria and previous | 46/1361 | 37/1367 | 1.19 | 0.77, 1.83 | 0.3557 |
| macrovascular disease without | | | | | |
| eGFR>45 mL/min/1.73 m2 | | | | | |
| Albuminuria and previous | 95/14612 | 108/1459 | 0.86 | 0.65, 1.14 | |
| macrovascular disease plus | | | | | |
| renal impairment (eGFR 15- | | | | | |
| <45 mL/min/1.73 m² with any | | | | | |
| UACR mg/g) | | | | | |
| Albuminuria and previous | 67/647 | 80/615 | 0.81 | 0.59, 1.12 | |
| macrovascular disease plus | | | | | |
| eGFR 45-75 mL/min/1.73 m² | | | | | |
| with an UACR >200 mg/g | | | | | |
| Established renal disease^{f} | | | | | |
| Yes | 162/2109 | 188/2074 | 0.84 | 0.68, 1.04 | 0.1510 |
| No | 47/1385 | 38/1411 | 1.19 | 0.78, 1.83 | |
| Established macrovascular | | | | | |
| disease and albuminuria | | | | | |
| Yes | 113/2008 | 117/1982 | 0.93 | 0.72, 1.20 | |
| No | 96/1486 | 109/1503 | 0.87 | 0.66, 1.15 | |
| Prevalent kidney disease | | | | | |
| (eGFR<60 mL/min/1.73 m² | | | | | |
| or macroalbuminuria | | | | | |
| UACR >300 mg/g) | | | | | |
| Yes | 191/2606 | 199/2541 | 0.91 | 0.75, 1.11 | 0.3918 |
| No | 18/887 | 27/944 | 0.69 | 0.38, 1.26 | |

Cox regression analysis in patients treated with ≥1 dose of study drug. Subgroup factors were pre-specified for the primary outcome. For the test of homogeneity of the treatment group difference among subgroups (test for group by covariate interaction) no adjustment for multiple tests were performed. NC: not calculated due to too few subgroup events
^{a}consistent results in the additional prespecifed age subgroups <65, 65-75 and >75 years (p-for interaction 0.9788),
^{b}an additional prespecifed regional subgroup analyses (Japan, non-Japan) involved too few events to be analysed,
^{c}additional prespecifed BP subgroups: SBP <140 and DBP <90 mmHg/SBP ≥140 or DBP ≥90 mmHg (p-for interaction 0.0060) and <160 and >=160 (p-for interaction 0.1113) mmHg,
^{d}consistent results in the additional prespecifed eGFR subgroups <60 and >=60 ml/min/1.73 m2 (p-for interaction 0.9339),
^{e}Per 2012 KDIGO criteria; Low risk defined as eGFR ≥60 ml/min/1.73 m² and UACR <30 mg/g, Moderately increased risk defined as eGFR 45-59 ml/min/1.73 m² and UACR <30 mg/g, or eGFR ≥60 ml/min/1.73 m² and UACR 30-300 mg/g, High risk defined as eGFR 30-44 ml/min/1.73 m² and UACR <30 mg/g, eGFR 45-59 ml/min/1.73 m² and UACR 30-300 mg/g, or eGFR ≥60 and UACR >300 mg/g, Very high risk defined as eGFR <30 ml/min/1.73 m² with any UACR, eGFR 30-44 and UACR 30-300 mg/g, or eGFR 45-59 ml/min/1.73 m² and UACR >300 mg/g,
^{f}patients in the “yes” category fulfils any one of the categories: albuminuria and previous macrovascular disease plus renal impairment (eGFR 15-<45mL/min/1.73 m² with any UACR), albuminuria and previous macrovascular disease plus renal impairment (eGFR 45-75 mL/min/1.73 m² with an UACR >200 mg/g), impaired renal function (eGFR 15-<45 mL/min/1.73 m² with any UACR mg/g), impaired renal function (eGFR 45-75 mL/min/1.73 m² with UACR >200 mg/g).

In yet further more detail with regard to the Cardiovascular and Renal Outcomes Trial:

Around three-quarters of patients in the Cardiovascular and Renal (Microvascular) Outcomes Trial had prevalent CKD at baseline, defined as reduced renal function (eGFR <60 mL/min/1.73 m2) and/or macroalbuminuria (urinary albumin-to-creatinine ratio >300 mg/g).

KDIGO categorises renal prognosis (for adverse kidney events) according to low, moderate, high and very high risk, based on a combination of albuminuria and renal risk. According to this internationally agreed standard, 44% of patients in the Cardiovascular and Renal Outcomes

Trial were at very high risk at baseline and a further 27% of patients were at high risk, with only 7% at low risk.

A limitation of dipeptidyl peptidase-4 (DPP-4) inhibitor cardiovascular outcomes trials (CVOTs) prior to the Cardiovascular and Renal Outcomes Trial is that only a minority of patients in the study cohorts had reduced renal function at baseline (estimated glomerular filtration rate (eGFR) <60 ml/min/1.73m2). Even fewer patients had severely reduced renal function (eGFR <30 ml/min/1.73m2) or macroalbuminuria (urinary albumin-to-creatinine ratio >300 mg/g). By contrast, 62% and 15% of patients in the Cardiovascular and Renal Outcomes Trial had reduced or severely reduced renal function at baseline, and the prevalence of macroalbuminuria was 39%, which compares with 10% of patients with macroalbuminuria at baseline in the saxagliptin CVOT. Macroalbuminuria prevalence for the sitagliptin CVOT was based on a limited number of patients for which data were available; prevalence of macroalbuminuria was not reported for the alogliptin CVOT.

The heart and kidneys are intricately linked by diverse interactions that drive a coincident morbidity between heart failure and chronic kidney disease (CKD). Hospitalization for heart failure (HHF) risk is elevated in patients presenting with impaired renal function (as measured by eGFR). However, linagliptin did not affect the risk of HHF, regardless of baseline renal function.

People with type 2 diabetes (T2D) with concomitant chronic kidney disease (CKD) and cardiovascular (CV) disease are at increased risk for recurrent CV events and hypoglycemia. Treatment of these individuals is clinically challenging, where the evidence-base for safety and efficacy of glucose lowering drugs is scarce, in particular in GFR categories G3b (eGFR 30-44 ml/min/1.73m²), G4 (eGFR <30) and G5 (eGFR <15). We analyzed baseline characteristics and effects on CV and kidney outcomes with the DPP-4 inhibitor linagliptin (LINA) vs. placebo (PBO), across GFR categories in the Cardiovascular and Renal Outcomes Trial. People with T2D and either i) UACR >30 mg/g with concomitant CV disease, or ii) eGFR <45 ml/min/1.73m² regardless of UACR, or eGFR ≥45-75mL/min/1.73m² and UACR >200 mg/g, were randomized to LINA 5 mg or placebo (PBO) q.d. in a double-blind fashion. The primary outcome was first occurrence of CV death, non-fatal myocardial infarction, or non-fatal stroke (3P-MACE), with an adjudicated secondary composite outcome of ESKD, renal death, or sustained ≥40% decrease in eGFR from baseline. Other adjudicated outcomes included hospitalized heart failure (hHF) and the 3P-MACE components. Subgroup-effects across GFR categories (G≤2, G3a, G3b and G≥4) were also assessed. Of the 6979 participants, 15.2% were in GFR category G≥4, 27.8% G3b, 19.3% G3a, and 37.7% G≤2 at baseline. Participants in G≥4 (mean±SD eGFR 23.4+4.2 mL/min/1.73m²) or G3b (eGFR 37.2±4.1) as compared with G3a (eGFR 51.4±4.4) and G≤2 (eGFR 81.6±16.7) had more albuminuria, longer T2D duration and were more frequently treated with insulin, but less often with sulfonylureas and metformin. Over a median 2.2 years, LINA did not affect the risk for 3P-MACE (HR.1.02 [95% CI, 0.89, 1.17]), the secondary kidney composite outcome (1.04 [0.89, 1.22]), hHF (0.90 [0.74, 1.08]), or CV mortality (0.96 [0.81, 1.14]).

Progression of albuminuria category (i.e. change from normal albuminuria to micro-/macroalbuminuria, or change from microalbuminuria to macroalbuminuria), occurred less frequently in the linagliptin (763/2162 [35.3%],) than in the placebo group (819/2129 [38.5%]); HR 0.86 (95% CI 0.78, 0.95), p=0.003.

Incidences were higher by declining kidney function, e.g. the 3P-MACE PBO incidence rate was 2.4 fold higher in G≥4 (9.6 per 100 patient-yrs) relative to G≤2 (4.0 per 100-patient yrs), whereas the kidney composite 9.8 fold (14.7 vs 1.5 per 100 patient-yrs), hHF 4.1 fold (6.2 vs 1.5 per patient-yrs) and CV death 3.0 fold (6.8 vs 2.3 per 100 patient-yrs) higher, respectively.

A consistent neutral effect was observed across all GFR categories (interaction p-values: 0.8 [3P-MACE], 0.36 [kidney composite], 0.88 [hHF], 0.23 [CV mortality]).

Progression of albuminuria was significantly reduced with linagliptin versus placebo overall and a consistent beneficial effect was observed across all eGFR categories (interaction p-value: 0.35).

Adverse events (AE) increased with declining kidney function, but the proportion with ≥1 AE, or ≥1 serious AE were balanced between LINA and PBO across the GFR categories. HbA1c was reduced significantly, but without increased risk for hypoglycemia with LINA vs PBO, across all GFR categories.

Among adults with T2DM and high CV and renal risk, the use of linagliptin compared with placebo, each added to usual care, over a median of 2.2 years resulted in a non-inferior risk of a composite CV outcome with no effect on the secondary kidney outcome.

In this patient population at very high risk for hHF and its complications, linagliptin can be used without increasing the risk for hHF.

These findings in a large, international Cardiovascular (Safety) and Renal (Microvascular) Outcomes Trial in patients with T2D and concomitant CV and renal disease support the safety and tolerability of LINA as a T2D therapy that can be used across a broad range of kidney disease, even including clinically challenging patients (with high cardiorenal risk), where the evidence-base for safety and efficacy of glucose lowering drugs is scarce, in particular in of GFR categories G3b (eGFR 30-44 ml/min/1.73m²), G4 (eGFR <30) and G5 (eGFR <15).

1. A method for treating a type 2 diabetes patient without increasing the risk of three point major adverse cardiovascular events (3P-MACE), comprising administering linagliptin, optionally in combination with one or more other active agents, to a patient in need thereof, wherein treatment of said patient with linagliptin does not increase the risk of one or more 3P-MACE compared to a patient treated with placebo, wherein the 3P-MACE is selected from the group consisting of cardiovascular death, nonfatal myocardial infarction (MI) and nonfatal stroke.

2. The method according to claim 1, wherein the method results in a hazard ratio (HR) of 1.02 (95% CI; 0.89, 1.17) for the risk of three point major adverse cardiovascular events (3P-MACE) by treatment with linagliptin relative to treatment with placebo.

3. A method for treating a type 2 diabetes patient without increasing the risk of hospitalization for heart failure, comprising administering linagliptin, optionally in combination with one or more other active agents, to a patient in need thereof, wherein treatment of said patient with linagliptin does not increase the risk of hospitalization for heart failure compared to a patient treated with placebo.

4. The method according to claim 3, wherein the method results in a hazard ratio (HR) of 0.90 (95% CI; 0.74, 1.08) for the risk of hospitalization for heart failure by treatment with linagliptin relative to treatment with placebo.

5. A method for treating a type 2 diabetes patient without increasing the risk of renal outcome events, comprising administering linagliptin, optionally in combination with one or more other active agents, to a patient in need thereof, wherein treatment of said patient with linagliptin does not increase the risk of one or more renal outcome events compared to a patient treated with placebo, wherein the renal outcome event is selected from the group consisting of renal death, sustained end stage renal disease (ESRD) and sustained decrease of 40% or more in estimated glomerular filtration rate (eGFR).

6. The method according to claim 5, wherein method results in a hazard ratio (HR) of 1.04 (95% CI; 0.89, 1.22) for the risk of renal outcome events by treatment with linagliptin relative to treatment with placebo.

7. A method for preventing, delaying the occurrence of, or reducing the risk of albuminuria progression in a type 2 diabetes patient, the method comprising administering linagliptin, optionally in combination with one or more other active agents, to a patient in need thereof, wherein treatment of said patient with linagliptin prevents, delays the occurrence of, or reduces the risk of albuminuria progression compared to a patient treated with placebo, wherein the albuminuria progression is selected from the group consisting of change from normal albuminuria to micro-or macroalbuminuria and change from microalbuminuria to macroalbuminuria.

8. A method for preventing, delaying the occurrence of, or reducing the risk of microvascular renal and/or eye complications in a type 2 diabetes patient, the method comprising administering linagliptin, optionally in combination with one or more other active agents, to a patient in need thereof, wherein treatment of said patient with linagliptin prevents, delays the occurrence of, or reduces the risk of one or more microvascular renal and/or eye complications compared to a patient treated with placebo, wherein the microvascular renal and/or eye complication is selected from the group consisting of renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and diabetes-related-blindness.

9. The method according to claim 1, wherein the patient is exposed to linagliptin treatment, optionally in combination with one or more other active agents, for at least 1.8 years or at least 1.9 years, and/or followed for at least 2.2 years.

10. The method according to claim 1, wherein the patient is at high or increased vascular risk of cardiovascular and/or renal complications or events.

11. The method according to claim 10, wherein the risk is based on history of established macrovascular disease and/or renal disease.

12. The method according to claim 1, wherein the patient has evidence of prevalent kidney disease or compromised kidney function, with or without macrovascular (cardiovascular) disease, as defined by i) albuminuria and previous macrovascular disease and/or ii) impaired renal function with predefined urine albumin creatinine ratio (UACR).

13. The method according to claim 1, wherein the patient has:
(i) albuminuria (micro or macro), defined as urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours), and previous macrovascular disease, defined as one or more of a) to f):
a) previous myocardial infarction,
b) advanced coronary artery disease,
c) high-risk single-vessel coronary artery disease,
d) previous ischemic or haemorrhagic stroke,
e) presence of carotid artery disease,
f) presence of peripheral artery disease;
and/or
(ii) impaired renal function with or without cardiovascular co-morbidities, defined by:
impaired renal function with an estimated glomerular filtration rate (eGFR) 15-45 mL/min/1.73 m² with any urine albumin creatinine ratio (UACR), or
impaired renal function with an estimated glomerular filtration rate (eGFR) ≥45-75 mL/min/1.73 m² with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24 hours).

14. The method according to claim 1, further comprising identifying the patient at high or increased risk of cardiovascular and/or renal events, prior to treatment with linagliptin.

15. The method according to claim 1, further comprising identifying the patient at risk of heart failure, prior to treatment with linagliptin.

16. The method according to claim 14, wherein the risk is based on history of established macrovascular disease and/or renal disease.

17. The method according to claim 14, wherein the risk is based on evidence of prevalent kidney disease or compromised kidney function, with or without macrovascular (cardiovascular) disease, as defined by i) albuminuria and previous macrovascular disease and/or ii) impaired renal function with predefined urine albumin creatinine ratio (UACR).

18. The method according to claim 14, wherein the risk is as defined by:
i) albuminuria (micro or macro), defined as urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours), and previous macrovascular disease, defined as one or more of a) to f):
a) previous myocardial infarction,
b) advanced coronary artery disease,
c) high-risk single-vessel coronary artery disease,
d) previous ischemic or hemorrhagic stroke,
e) presence of carotid artery disease,
f) presence of peripheral artery disease;
and/or
(ii) impaired renal function with or without cardiovascular co-morbidities, defined by:
impaired renal function with an estimated glomerular filtration rate (eGFR) 15-4 5mL/min/1.73 m² with any urine albumin creatinine ratio (UACR), or
impaired renal function with an estimated glomerular filtration rate (eGFR) ≥45-75 mL/min/1.73 m² with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24hours).

19. The method according to claim 1, wherein the patient has
albuminuria, defined by microalbuminuria (UACR 30-300 mg/g) or macroalbuminuria (UACR >300 mg/g),
and/or
impaired renal function, defined by mild (eGFR ≥60 to <90 mL/min/1.73 m2), moderate (eGFR ≥45 to <60 mL/min/1.73 m2), moderate/severe (eGFR ≥30 to <45 mL/min/1.73 m2) or severe (eGFR <30 mL/min/1.73 m2) renal impairment.

20. A method for treating a type 2 diabetes patient at risk of heart failure, the method comprising treating the patient with linagliptin.

21. The method according to claim 20, wherein the treatment of said patient with linagliptin does not increase the risk of hospitalization for heart failure compared to a patient treated with placebo.

22. The method according to claim 20, further comprising identifying the patient at risk of heart failure prior to treatment with linagliptin.

23. A method of treating a type 2 diabetes patient who has high or increased risk for cardiovascular and/or renal events, the method comprising treating the patient with linagliptin.

24. The method according to claim 23, wherein the treatment of said patient with linagliptin
i) does not increase the risk of one or more three point major adverse cardiovascular events (3P-MACE), wherein the one or more three point major adverse cardiovascular events (3P-10 MACE) are selected from the group consisting of cardiovascular death, nonfatal myocardial infarction (MI) and nonfatal stroke,
ii) does not increase the risk of hospitalization for heart failure, and/or
iii) does not increase the risk of one or more renal outcome events, wherein the one or more renal outcome events are selected from the group consisting of renal death, sustained end stage renal disease (ESRD) and sustained decrease of 40% or more in estimated glomerular filtration rate (eGFR),
each compared to a patient treated with placebo.

25. The method according to claim 23, further comprising identifying the patient at high or increased risk for cardiovascular and/or renal events prior to treatment with linagliptin.

26. The method according to claim 22, wherein the risk is based on history of established macrovascular disease and/or renal disease,
such as defined by i) albuminuria and previous macrovascular disease and/or ii) impaired renal function with predefined urine albumin creatinine ratio (UACR), such as defined by
i) albuminuria (micro or macro), defined as urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours), and
previous macrovascular disease, defined as one or more of a) to f):
a) previous myocardial infarction,
b) advanced coronary artery disease,
c) high-risk single-vessel coronary artery disease,
d) previous ischemic or haemorrhagic stroke,
e) presence of carotid artery disease,
f) presence of peripheral artery disease;
and/or
(ii) impaired renal function with or without cardiovascular co-morbidities, defined by:
impaired renal function with an estimated glomerular filtration rate (eGFR) 15-45 mL/min/1.73 m2 with any urine albumin creatinine ratio (UACR), or
impaired renal function with an estimated glomerular filtration rate (eGFR) ≥45-75 mL/min/1.73 m2 with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24 hours).

27. The method according to claim 20, wherein the patient has
albuminuria, defined as microalbuminuria (UACR 30-300 mg/g) or macroalbuminuria (UACR >300 mg/g),
and/or
impaired renal function, defined as mild (eGFR ≥60 to <90 mL/min/1.73 m2), moderate (eGFR ≥45 to <60 mL/min/1.73 m2), moderate/severe (eGFR ≥30 to <45 mL/min/1.73 m2) or severe (eGFR <30 mL/min/1.73 m2) renal impairment.

28. The method according to claim 1, wherein linagliptin is administered in an oral daily dose of 5 mg.
